(19) United States
(12) Patent Application Publication
(10) Pub. No.: US 2003/0040009 A1
(43) Pub. Date: Feb. 27, 2003

(54) Saliva-based methods for preventing and assessing the risk of diseases

(75) Inventors: Paul C. Denny, Rossmoor, CA (US); Mahvash Navazesh, Los Angeles, CA (US); Patricia A. Denny, Rossmoor, CA (US)

Correspondence Address: Rajiv Yadav, McCutchen, Doyle, Brown & Emersen, LLP, 18th Floor, Three Embarcadero Center, San Francisco, CA, 94111, US

(73) Assignee: University of Southern California

(21) Appl. No.: 09/929,293

(22) Filed: Aug. 14, 2001

(51) Int. Cl. G01N 33/53; G01N 33/554; G01N 33/569

(52) U.S. Cl. 435/7.1; 435/7.32

(57) ABSTRACT

This present invention provides compositions and methods for assessing the risk of a disease using salivary analysis. Specifically, the present invention provides a method for predicting the risk of a disease, comprising the steps of: a) providing a saliva sample from a subject; b) isolating a mucin in the saliva sample to produce an isolated mucin; and c) quantitating the content of a component in the isolated mucin, to predict the risk of a disease in the subject. The present invention also provides methods for reducing the risk of a disease and diagnostic kits for detecting a disease based on measurement of the content of a component in a salivary mucin.

### FIELD OF THE INVENTION

This present invention provides compositions and methods for assessing the risk of a disease using salivary analysis.

### BACKGROUND OF THE INVENTION

Insight into a wide range of clinical situations may be gained from salivary analysis. (See e.g., Mandel, I. D., “Salivary Diagnosis: Promises, Promises,” in Saliva as a Diagnostic Fluid, Malamud et al., eds., Ann. NY Acad. Sciences 694: 1-10 (1993)). The earliest sialochemical studies on oral fluids examined saliva for specific components that would be diagnostic for various systemic conditions, including gout and rheumatism. (Atkinson et al., “Guidelines for Saliva Nomenclature and Collection,” in Saliva as a Diagnostic Fluid, Ann. NY Acad. Sciences 694: xi-xii (1993)). A recent example utilizes salivary acetaminophen concentration to assess the gastric emptying rate of liquids. A number of studies report using saliva specimens for monitoring pharmaceuticals and chemicals, including taxol (Svojanovsky et al., “High sensitivity ELISA determination of taxol in various biological fluids,” J. Pharm. Biomed. Anal. 20: 549-555 (1999)); caffeine (Akinyinka et al., “The effects of acute falciparum malaria on the disposition of caffeine and the comparison of saliva and plasma-derived pharmacokinetic parameters in adult Nigerians,” Eur. J. Clin. Pharmacol. 56: 159-165 (2000)); and the protease inhibitor, indinavir (Wintergerst et al., “Use of saliva specimens for monitoring indinavir therapy in human immunodeficiency virus-infected patients,” Antimicrobial Agents and Chemotherapy 44: 2572-2574 (2000)).

In general, the analysis of saliva for diagnostic purposes has been directed towards evaluating systemic disease (e.g., Sjögren's syndrome, cystic fibrosis, HIV infection, etc.), or as a means of determining systemic levels of therapeutic drugs (e.g., steroids). (See e.g., Ferguson, “Current diagnostic uses of saliva,” J. Dent. Res. 66(2): 420-424 (1987). There have also been many attempts to measure other factors in saliva and then relate them to oral diseases. For example, salivary analysis has also been used to diagnose periodontal disease (See e.g., U.S. Pat. No. 6,063,588 to Lamster; U.S. Pat. No. 5,376,532 to Singer, Jr.; U.S. Pat. Nos. 5,866,432 and 5,736,341 to Sorsa et al.; and U.S. Pat. No. 5,756,361 to Winterbottom et al.). However, the vast majority of studies have not been able to relate factors in saliva with other common oral diseases such as dental caries. These studies reported values for pH, various ions, macromolecules, and flow rate, but found little evidence of a correlation. The few studies that have shown a small amount of correlation were found not to correlate with other studies. Thus, there remains a need for a saliva-based test for predicting the risk of oral diseases and associated diseases that is simple and accurate.

### SUMMARY OF THE INVENTION

It is an object of the present invention to provide compositions and methods for predicting and reducing the risk of a disease using salivary analysis. It is not intended that the present invention be limited to compositions and methods for predicting and preventing specific diseases. Thus, the present invention provides methods for predicting and reducing the risk of a disease and diagnostic kits for detecting a disease based on measurement of the content of a component in a salivary mucin.

The present invention provides a method for predicting the risk of a disease, comprising the steps of: a) providing a saliva sample from a subject; b) isolating a mucin in the saliva sample to produce an isolated mucin; and c) quantitating the content of a component in the isolated mucin, to predict the risk of a disease in a subject. The methods of the present invention can further comprise the step of reporting the content of the component in the isolated mucin as units per milliliter of the saliva sample. The saliva sample may be a stimulated saliva sample or, in a preferred embodiment, an unstimulated saliva sample. The salivary mucin may be any of the salivary mucins. In preferred embodiments, the mucin is MUC5AC mucin, MUC5B mucin or MUC7 mucin. In preferred embodiments, the component of the mucin is the total apomucin, the total carbohydrate or the sialic acid comprising the mucin.

The methods of the present invention can also further comprise the step of assessing the risk of the disease as high, medium or low. The methods of the present invention can also further comprise the step of assessing the risk of future development of a disease in a subject. In one embodiment, the methods of the present invention comprise the step of assessing the risk of future development of a disease in a subject at subsequent ages.

In one embodiment, the isolating step comprises isolating the mucin using SDS-PAGE. In yet another embodiment, the quantitating step comprises specifically binding the component of the isolated mucin. The specific binding can comprise direct binding or facilitated binding. In one embodiment, the direct binding comprises directly binding using a dye. In another embodiment, the facilitated binding comprises linking a specifically binding member pair to a surface that specifically binds to the component of the isolated mucin. In one embodiment, the specifically binding member pair is selected from the group consisting of antibodies and lectins.

Furthermore, the present invention provides a method for predicting the risk of a disease in a human. The human can be selected from the group consisting of males and females. The age of the human can be between 18 and 35 years old; between 2 and 45 years old; between 2 and 80 years old or above; or between 15 and 60 years old or above. It is not intended that the compositions and methods of the present invention be limited to predicting diseases of human subjects within a particular age group.

The present invention also provides a diagnostic kit for detecting a disease, comprising: a) a means for collecting a saliva sample; b) a means for isolating a mucin in the saliva sample, to produce an isolated mucin; c) a means for measuring the amount of component in the isolated mucin; and d) an oral fluid standard for comparing the amount of the component in the isolated mucin. The means for isolating the mucin from other in the saliva can comprise an SDS-PAGE gel. The saliva sample may be a stimulated saliva sample or, in a preferred embodiment, an unstimulated saliva sample. The salivary mucin may be any of the salivary mucins. In preferred embodiments, the mucin is MUC5AC mucin, MUC5B mucin or MUC7 mucin. In preferred embodiments, the component of the mucin is the total apomucin, the total carbohydrate or the sialic acid comprising the mucin.

The diseases being predicted include, but are not limited to, oral diseases and associated medical disorders. Oral diseases and associated medical disorders include, but are not limited to, dental caries; periodontal diseases (e.g., gingivitis, adult periodontitis, early-onset periodontitis, etc.); diseases associated with periodontal disorders (e.g., pulmonary and respiratory diseases, and cardiovascular diseases such as heart attack, stroke, atherosclerosis, etc.); diabetes; perinatal disorders (e.g., low birth weight and premature births); mucosal infections; oral and pharyngeal cancers; precancerous lesions; associated autoimmune disorders (e.g., Sjörgren's syndrome); HIV; and osteoporosis.

Mucosal infections include, but are not limited to, oral candidiasis, herpes simplex virus infections (types 1 and 2) (e.g., cold sores, genital herpes, etc.), herpes zoster virus infections (shingles), varicella zoster virus infections (chicken pox), human papillomavirus infections (e.g., genital warts, condyloma acuminatum, etc.), oral human papillomavirus infections, and recurrent aphtous ulcers.

In a preferred embodiment, the present invention provides a method for predicting dental caries risk. The dental caries can be early-onset dental caries, adult dental caries, root caries, DFT, DMF, or DMFS.

The present invention also provides a method for preventing the risk of a disease, comprising the steps of: a) providing a saliva sample from a subject; b) isolating a mucin in the saliva sample to produce an isolated mucin; c) quantitating the content of a component in the isolated mucin; and d) administering a therapeutic reagent for treating the disease in a subject when the content of the component in the isolated mucin significantly falls below the level expressed in an oral fluid standard. The oral fluid standard can comprise a sample from a normal control (i.e., a subject who does not suffer from the disease being tested for). The saliva sample may be a stimulated saliva sample or, in a preferred embodiment, an unstimulated saliva sample. The salivary mucin may be any of the salivary mucins. In preferred embodiments, the mucin is MUC5AC mucin, MUC5B mucin or MUC7 mucin. In preferred embodiments, the component of the mucin is the total apomucin, the total carbohydrate or the sialic acid comprising the mucin.

In one embodiment, the isolating step comprises isolating the mucin using SDS-PAGE. In yet another embodiment, the quantitating step comprises specifically binding the component of the isolated mucin. The specific binding can comprise direct binding of or facilitated binding. In one embodiment, the direct binding comprises directly binding using a dye. In another embodiment, the facilitated binding comprises linking a specifically binding member pair to a surface that specifically binds to the component of the isolated mucin. In one embodiment, the specifically binding member pair is selected from the group consisting of antibodies and lectins.

In a particular embodiment, the present invention provides a method for preventing the risk of a disease in a human. The human can be selected from the group consisting of males and females. The age of the human can be between 18 and 35 years old; between 2 and 45 years old; between 2 and 80 years old or above; or between 15 and 60 years old or above. It is not intended that the compositions and methods of the present invention be limited to preventing diseases of human subjects within a particular age group.

The disease being prevented includes, but is not limited to, oral diseases and associated medical disorders. Oral diseases and associated medical disorders include, but are not limited to, dental caries; periodontal diseases (e.g., gingivitis, adult periodontitis, early-onset periodontitis, etc.); diseases associated with periodontal disorders (e.g., pulmonary and respiratory diseases, and cardiovascular diseases such as heart attack, stroke, atherosclerosis, etc.); diabetes; perinatal disorders (e.g., low birth weight and premature births); mucosal infections; oral and pharyngeal cancers; precancerous lesions; associated autoimmune disorders (e.g., Sjörgren's syndrome); HIV; and osteoporosis.

Mucosal infections include, but are not limited to, oral candidiasis, herpes simplex virus infections (types 1 and 2) (e.g., cold sores, genital herpes, etc.), herpes zoster virus infections (shingles), varicella zoster virus infections (chicken pox), human papillomavirus infections (e.g., genital warts, condyloma acuminatum, etc.), oral human papillomavirus infections, and recurrent aphtous ulcers.

In a preferred embodiment, the present invention provides a method for preventing dental caries risk. The therapeutic agent to be administered is an anti-caries agent. The dental caries can be early-onset dental caries, adult dental caries, root caries, DFT, DMF, or DMFS.

### BRIEF DESCRIPTION OF THE FIGURES

FIG. 1 describes a linear regression analysis for the relationship of MUC7 mucin concentration in unstimulated saliva with DMF in a group of 20 young adults.

FIG. 2 describes a linear regression analysis for the relationship of MUC7 mucin concentration in unstimulated saliva with DFT in the 20 adults.

FIG. 3 describes the corresponding scatter plot for the data provided in Table 2.

FIG. 4 describes a linear regression analysis for the relationship of MUC7 mucin concentration in unstimulated saliva with DFT for 16 young adults after removing the four outliers shown in FIG. 3.

FIG. 5 describes a linear regression analysis for the relationship of MUC7 mucin concentration in unstimulated saliva with DFT for the four outliers shown in FIG. 3.

FIG. 6 describes a scatter diagram of mucin concentration versus DFT with the group of 16 and the four member outlier group represented separately.

FIG. 7 describe a regression line of mucin concentration versus DFT with representative 95% confidence levels for DFT predicted by either one or four saliva collections based on individual CVs (coefficients of variation).

FIG. 8 describes significant ranges of DFT prediction by MUC7 mucin concentration based solely on 95% confidence intervals of the regression equation.

FIG. 9 describes a preferred model for predicting caries experience based on a combination of variation of MUC7 mucin concentration in individual subjects and the 95%confidence interval of the regression equation, according to one embodiment of the present invention.

Definitions

To facilitate understanding of the invention, a number of terms are defined below.

As used herein, the term “saliva” refers to an oral fluid, regardless of where the saliva is secreted in the oral cavity, or how it is collected.

In a preferred embodiment, the sample of saliva is unstimulated. As used herein, the term “unstimulated saliva” means that the subject will expectorate in a collection vessel without stimulation of salivary flow. For example, a subject's saliva may not be stimulated by chewing on a piece of paraffin film or tart candy.

As used herein, the terms “prediction of dental caries risk” or “prediction of dental caries experience” refer to the risk of future dental caries development and the forecast of the current accumulated number of caries and fillings, respectively. Caries is a disease characterized by demineralization of the dental enamel and of the dentin in various stages of progress, until it affects the pulp space. Fillings refer to thoses caries that have been treated or restored. Prediction is synonymous with the terms, prognostication, forecasting, foretelling, foreseeing, portending, etc.

As used herein, the term “oral fluid standard” refers to a solution useful as a surrogate for naturally occurring oral fluid in the testing, calibration and standardization of oral fluid collection methods and devices, oral fluid handling, preservation and storage methods and devices, and oral fluid-based assay methods and devices. Oral fluid standards are not intended as an in vivo therapeutic replacement or supplement for saliva, but rather are used as ex vivo testing standards. The term oral fluid standard may refer to the oral fluid surrogate composition alone, or to the oral fluid surrogate spiked with one or more additional components such as an analyte and/or human serum. The particular meaning of the term oral fluid standard will be apparent from the context in which it is used.

As used herein, the term “oral fluid” refers to one or more fluids found in the oral cavity individually or in combination. These include, but are not limited to saliva and mucosal transudate. It is recognized that oral fluids (e.g., saliva) are a combination of secretions from a number of sources (e.g., parotid, submandibular, sublingual, accessory glands, gingival mucosa and buccal mucosa), and the term oral fluid includes the secretion of each of these sources individually or in combination.

As used herein, the term “mucins” refers to acid mucopolysaccharides complexed with proteins. The acid mucopolysaccharides are a group of related heteropolysaccharides usually containing two types of alternating monosaccharide units, of which at least one has an acidic group (typically either a carboxyl or a sulfuric group). The term “MUC7” refers to a particular mucin gene. The term “MUC7 mucin or occasionally MUC7 protein” refers to the protein encoded by the MUC7 gene and posttranslationally modified to contain the necessary carbohydrates and possibly sulphur to qualify it as a mucin, which is a recognized biochemical class of glycoproteins.

As used herein, the term “specific binding member pair” means a molecule which is one of two different molecules, having an area which specifically binds to and is thereby defined as being complementary with another molecule. The two molecules are related in the sense that their binding to each other is such that they are capable of distinguishing their binding partner from other assay constituents having similar characteristics. The members of the specific binding pair may be referred to as ligand and receptor. Specific binding pair members include, but are not limited to, immunological binding pairs such as an antigen-antibody binding pair, and other non-immunological specific binding pairs, such as biotin-avidin, hormone-hormone receptor, nucleic acid-duplexes, etc. As used herein, the term “ligand” refers to any compound for which a receptor exists, either naturally or synthetically.

As used herein, the term “antibody” means an immunoglobulin having an area that specifically binds to, and is thereby defined as complementary with another molecule. The antibody can be polyclonal or monoclonal. Antibodies may include a complete immunoglobulin or fragments thereof, or various immunoglobulin classes and isotypes.

As used herein, the term “antigen” refers to any compound capable of binding to an antibody, or against which antibodies may be raised.

As used herein, the term “subject” refers to a subject whose saliva is being tested for a particular disease. The subject can be a human or an animal.

As used herein, the terms “normal subject” or “normal control” refer to a subject who does not suffer from the particular disease being tested for (e.g., dental caries or any diseases associated with dental caries experience).

As used herein, the term “SDS-PAGE” refers to “sodium dodecyl sulfate-polyacrylamide gel.” SDS-PAGE is a variation of the protein separation technique, PAGE (“polyacrylamide gel electrophoresis), which separates proteins on the basis of their charge to mass ratio by applying an electric field to a protein mixture. Unlike PAGE, SDS-PAGE separates proteins according to mass by conferring upon the proteins a negative charge proportional to mass.

As used herein, the terms “oral disorders” and “oral diseases” refer to diseases and disorders affecting the oral cavity, and associated medical disorders. Oral disorders include, but are not limited to, dental caries; periodontal diseases (e.g., gingivitis, adult periodontitis, early-onset periodontitis, etc.); mucosal infections (e.g., oral candidiasis, herpes simplex virus infections, oral human papillomavirus infections, recurrent aphtous ulcers, etc.); oral and pharyngeal cancers; and precancerous lesions.

As used herein, the term “associated medical disorders” refers to medical conditions associated with periodontal diseases (e.g., pulmonary and respiratory diseases, and cardiovascular diseases such as heart attack, stroke, atherosclerosis, etc.); associated autoimmune disorders (e.g., Sjörgren's syndrome); HIV; and osteoporosis.

### DETAILED DESCRIPTION OF THE INVENTION

The present invention provides compositions and methods for assessing the risk of a disease using salivary analysis. In particular, the compositions and methods of the present invention can be used to predict and prevent the risk of oral diseases and other associated diseases.

I. Oral Diseases and Associated Diseases

The most common oral diseases are dental caries and the periodontal diseases. Individuals are vulnerable to dental caries throughout life, affecting 85 percent of adults aged 18 and older. Periodontal diseases are most often seen in maturity, with the majority of adults experiencing some signs and symptoms by the mid-30s. Certain rare forms of periodontal disease affect young people. Oral disorders also include certain mucosal infections, as well as oral and pharyngeal cancers and precancerous lesions.

A. Dental Caries

Caries is a unique multifactorial infectious disease. (Lenander-Lumikari et al., “Saliva and Dental Caries,” Adv. Dent. Res. 14: 40-47 (December 2000)). Dental caries affects teeth at all levels and can cause extensive crown mutilations, bacterial disorders of the periapical tissues, or even loss of the affected dental elements. Clinically, the disease is characterized by demineralization of the dental enamel and of the dentin in various stages of progress, until it affects the pulp space. When the lesion passes beyond the enamel-dentin border, a phlogistic reaction of the pulp tissues is constantly observed, with the formation of reaction dentin in some cases. Approximately 50% of adult individuals have at least four caries-related lesions that are treated and require treatment, and approximately 30% of adult individuals have over 50% of their teeth affected by caries. (See U.S. Pat. No. 5,830,489 to Valenti et al.).

The bacterium Streptococcus mutans, or S. mutans, is known to be a prime etiologic agent for the initiation and progression of human dental caries, or cavities. S. mutans is one of the primary factors in acid dissolution of the apatite (mineral) component of the enamel then the dentin, or of the cementum then the dentin. (Tanzer, J. M., “Understanding dental caries: an infectious disease, not a lesion,” Inter. J. Oral Biol. 22:205-214 (1997)). A strong correlation between the proportion of S. mutans in dental plaque or in saliva relative to other bacterial species and the presence or risk of future outbreaks of dental caries has been documented. (Tanzer, J. M., supra). Therefore, S. mutans in plaque or saliva may serve as an index for both caries activity state and caries risk or susceptibility. These indices play an increasingly important role in the diagnosis and treatment of dental caries. (Hume, W. R., “Need for change in standards of caries diagnosis-perspective based on the structure and behavior of the carious lesion,” J. Dent. Educ. 57: 439-443 (1993)).

Present techniques for detecting and quantitatively determining S. mutans include bacterial culture with selective media using either broth or agar plate systems, and polymerase chain reaction techniques. (Ellen, R. P., Microbiological assays for dental caries and periodontal disease susceptibility,” Oral Sci. Rev. 8: 3-23 (1976); Igarashi et al., “Direct detection of Streptococcus mutans in human dental plaque by polymerase chain reaction,” Oral Microbiol. and Immunol. 11: 294-298 (1996); U.S. Pat. No. 5,374,538 to Bratthall; U.S. Pat. No. 4,692,407 to Jordan et al.). However, each of these methods require significant time (on the order of days), well trained personnel and sophisticated equipment to perform. Consequently, existing techniques are relatively expensive and time consuming. Moreover, the use of the titer of S. mutans in the oral cavity as a predictor of caries risk is consistently significant only within the first two years of age.

Human dental caries may also be detected by changes in translucency, color, hardness or X-ray density of teeth. However, these technologies have limitations both in specificity and reproducibility. Furthermore, they do not show whether or not the disease is active at a single time point. (U.S. Pat. No. 6,231,857 to Shi et al.).

B. Periodontal Diseases

Like dental caries, the periodontal diseases are infections caused by bacteria in the biofilm (dental plaque) that forms on oral surfaces. The basic division in the periodontal diseases is between gingivitis, which affects the gums, and periodontitis, which may involve all of the soft tissue and bone supporting the teeth. Gingivitis and milder forms of periodontitis are common in adults. The percentage of individuals with moderate to severe periodontitis, in which the destruction of supporting tissue may cause the tooth to loosen and fall out, increases with age.

Gingivitis

Gingivitis is an inflammation of the gums characterized by a change in color from normal pink to red, with swelling, bleeding, and often sensitivity and tenderness. These changes result from an accumulation of biofilm along the gingival margins and the immune system's inflammatory response to the release of destructive bacterial products. The early changes of gingivitis are reversible with thorough toothbrushing and flossing to reduce plaque. Without adequate oral hygiene, however, these early changes can become more severe, with infiltration of inflammatory cells and establishment of a chronic infection. Biofilm on tooth surfaces opposite the openings of the salivary glands often mineralizes to form calculus or tartar, which is covered by unmineralized biofilm—a combination that may exacerbate local inflammatory responses (Mandel, J. Am. Dent. Assoc. 126: 573-80 (1995)). A gingival infection can persist for months or years, yet never progress to periodontitis.

Gingival inflammation does not appear until the biofilm changes from one composed largely of gram-positive streptococci (which can live with or without oxygen) to one containing gram-negative anaerobes (which cannot live in the presence of oxygen). Numerous attempts have been made to pinpoint which microorganisms in the supragingival (above the gum line) plaque are the culprits in gingivitis. Frequently mentioned organisms include Fusobacterium nucleatum, Veillonella parvula, and species of Campylobacter and Treponema.

Gingival inflammation may be influenced by steroid hormones, occurring as puberty gingivitis, pregnancy gingivitis, and gingivitis associated with birth control medication or steroid therapy. The presence of steroid hormones in tissues adjacent to biofilm apparently encourages the growth of certain bacteria and triggers an exaggerated response to biofilm accumulation (Caton, “Periodontal diagnosis and diagnostic aids,” in Proceedings of the World Workshop in Clinical Periodontics, American Academy of Periodontology, pp. I-1-22, Princeton, N.J. (1989)). Certain prescription drugs may also lead to gingival overgrowth and inflammation. These include the antiepileptic drug phenytoin (DILANTIN®), cyclosporin, and various calcium channel blockers used in heart disease.

Adult Periodontitis

The most common form of adult periodontitis is described as general and moderately progressing. A second form is described as rapidly progressing and severe, and is often resistant to treatment. The moderately progressive adult form is characterized by a gradual loss of attachment of the periodontal ligament to the gingiva and bone, along with loss of the supporting bone. It is most often accompanied by gingivitis (Genco, “Classification of clinical and radiographic features of periodontal diseases,” in Contemporary Periodontics, Genco et al., eds., pp. 63-81, (1990)). It is not necessarily preceded by gingivitis, but the gingivitis-related biofilm often seeds the subgingival plaque. The destruction of periodontal ligament and bone results in the formation of a pocket between the tooth and adjacent tissues, which harbors subgingival plaque. The calculus formed in the pocket by inflammatory fluids and minerals in adjacent tissues is especially damaging (Mandel and Gaffar, J. Clin. Periodontol. 13: 249-57 (1986)).

The severity of periodontal disease is determined through a series of measurements, including the extent of gingival inflammation and bleeding, the probing depth of the pocket to the point of resistance, the clinical attachment loss of the periodontal ligament measured from a fixed point on the tooth (usually the cemento-enamel junction), and the loss of adjacent alveolar bone as measured by x-ray (Genco, J. Periodontol. 67(10 Suppl.): 1041-9 (1996)). Severity is determined by the rate of disease progression over time and the response of the tissues to treatment. Adult periodontitis often begins in adolescence but is usually not clinically significant until the mid-30s. Prevalence and severity increase but do not accelerate with age (Beck, Ann. Periodontol. 7(1): 322-57 (1996)).

Early-Onset Periodontitis

The forms of periodontitis occurring in adolescents and young adults generally involve defects in neutrophil function (Van Dyke et al., Infect. Immun. 27(1): 124-31 (1980)). Localized juvenile periodontitis (LJP) mainly affects the first molar and incisor teeth of teenagers and young adults, with rapid destruction of bone but almost no telltale signs of inflammation and very little supragingival plaque or calculus. Actinobacillus actinomycetemcomitans has been isolated at 90 to 100 percent of diseased sites in these patients, but is absent or appears in very low frequency in healthy or minimally diseased sites (Socransky and Haffajee, J. Periodontol. 63(4 Suppl.): 322-31 (1992)). It is possible that the bacteria are transmitted among family members through oral contacts such as kissing or sharing utensils, because the same bacterial strain appears in affected family members. However, evidence of a neutrophil defect argues for a genetic component. Another organism frequently associated with LJP is Capnocytophaga ochracea. Neither of these bacteria dominate in the generalized adult form of the disease, where Porphyromonas gingivalis is considered of greatest significance (Schenkein and Van Dyke, Periodontol. 6: 7-25 (1994).

Prepubertal periodontitis is rare and may be either general or localized. The generalized form begins with the eruption of the primary teeth and proceeds to involve the permanent teeth. There is severe inflammation, rapid bone loss, tooth mobility, and tooth loss. The localized form of the disease is less aggressive, affecting only some primary teeth. The infection involves many of the organisms associated with periodontitis, but the mix can differ somewhat, with Actinobacillus actinomycetemcomitans, Prevotella intermedia, Eikenella corrodens, and several species of Capnocytophaga implicated (Caton, supra). Defects in neutrophil function in both forms of the disease may explain why patients are highly susceptible to other infections as well (Suzuki, Dent. Clin. North Am. 32(2): 195-216 (1988)).

Other Diseases Associated With Oral Disorders

Chronic obstructive pulmonary disease, characterized by obstruction of airflow due to chronic bronchitis or emphysema and by recurrent episodes of respiratory infection, has been associated with poor oral health status (Hayes et al., Ann. Periodontol. 3(1): 257-61 (1998); Scannapieco et al., Ann. Periondontol. 3(1): 251-6 (1998)). A positive relationship between periodontal disease and bacterial pneumonia has also been shown. (Scannapieco and Mylotte, J. Periodontol 67(10 Suppl.): 1114-22 (1996)).

Recent studies have also underscored the association of oral infections with certain medically important conditions. Increasing data implicate periodontal disease as a risk factor for cardiovascular diseases such as heart attack and stroke. (See e.g., U.S. Pat. No. 6,130,042 to Diehl et al.; J. Beck et al., “Periodontal Disease and Cardiovascular Disease,” J. Periodontol. 67:1123 (1996)). Epidemiologic studies indicate that, even after accounting for other known risk factors for cardiovascular disease, the relative risk attributable to periodontal infections is significant. Secondly, recent studies have shown that mothers with periodontitis are at greater risk for having low weight babies than those without periodontitis. (See, Offenbacher et al., “Periodontal Infection as a Possible Risk Factor for Preterm Low Birth Weight,” J. Periodontol. 67:1103 (1996)).

There is also growing acceptance that diabetes is associated with increased occurrence and progression of periodontitis—so much so that periodontitis has been called the “sixth complication of diabetes” (Löe, Diabetes Care 16(1): 329-34 (1993)). The risk is independent of whether the diabetes is type 1 or type 2. Type 1 diabetes is the condition in which the pancreas produces little or no insulin. It usually begins in childhood or adolescence. In type 2 diabetes, secretion and utilization of insulin are impaired; onset is typically after age 30. Together, these two types of diabetes affect an estimated 15.7 million people in the United States and represent the seventh leading cause of death (National Institute of Diabetes and Digestive and Kidney Diseases (NIDDK). Diabetes statistics. NIH Pub. no. 99-3892 (1999)). The goal of diabetic care is to lower blood glucose levels to recommended levels. Some investigators have reported a two-way connection between diabetes and periodontal disease, proposing that not only are diabetic patients more susceptible to periodontal disease, but the presence of periodontal disease affects glycemic control.

Oral Disease and Adverse Pregnancy Outcomes

Preterm birth and low birth weight are considered the leading perinatal problems in the United States (Gibbs et al., Am. J. Obstet. Gynecol. 166(5): 1515-28 (1992)). Although infant mortality rates have decreased substantially over the past generation, the incidence of low birth weight (just under 300,000 cases in 1995) has not shown a comparable decline (Institute of Medicine, Committee to Study the Prevention of Low Birth Weight, Division of Health. Promotion and disease progression. Preventing low birth weight. Washington: National Academy Press (1985)). Over 60 percent of the mortality of infants without structural or chromosomal congenital defects may be attributed to low birth weight (Shapiro et al., Am. J. Obstet. Gynecol. 136(3): 363-73 (1980)).

Oral disease may contribute to adverse outcomes of pregnancy as a consequence of a chronic oral inflammatory bacterial infection. For example, toxins or other products generated by periodontal bacteria in the mother can reach the general circulation, cross the placenta, and harm the fetus. In addition, the response of the maternal immune system to the infection elicits the continued release of inflammatory mediators, growth factors, and other potent cytokines, which may directly or indirectly interfere with fetal growth and delivery.

Evidence of increased rates of amniotic fluid infection, chorioamnion infection, and histologic chorioamnionitis supports an association between preterm birth, low birth weight, and general infection during pregnancy. It is noteworthy that the largest proportion of such infections occurred during the pregnancies of the most premature births (Hillier et al., N. Engl. J. Med. 319(15): 972-8 (1988); Hillier et al., N. Engl. J. Med. 333(26): 1737-42 (1995)). The biological mechanisms involve bacteria-induced activation of cell-mediated immunity leading to cytokine production and the synthesis and release of prostaglandins, which may trigger preterm labor (Hillier et al., supra). Elevated levels of prostaglandin as well as cytokines (interleukin-1 (IL-1), interleukin-6 (IL-6), and tumor necrosis factor alpha (TNF-α) have been found in the amniotic fluid of patients in preterm labor with amniotic fluid infection (Romero et al., Am. J. Obstet. Gynecol. 186(6 Pt 1): 1654-64 (1993)), compared with levels in patients with preterm labor without infection.

C. Mucosal Infections

The mucosal lining of the mouth is subject to a variety of infections and conditions, ranging from benign canker sores to often fatal cancers.

Oral Candidiasis

Chronic hyperplastic candidiasis is a red or white lesion that may be flat or slightly elevated and may adhere to soft or hard tissue surfaces, including dental appliances. It is caused by species of Candida, especially Candida albicans, the most common fungal pathogen isolated from the oral cavity. Normally, the fungi are present in relatively low numbers in up to 65 percent of healthy children and adults and cause no harm (McCullough et al., Int. J. Oral Maxillofac. Surg. 25: 136-44 (1996)).

The most common form of oral candidiasis is denture stomatitis. It occurs when tissues are traumatized by continued wearing of ill-fitting or inadequately cleaned dental appliances and is described as chronic erythematous candidiasis. Another form, candidal angular cheilosis, occurs in the folds at the angles of the mouth and is closely associated with denture sore mouth (Tyldesley and Field, Oral medicine, 4^{th} ed., Oxford University Press (1995)). Other common forms of Candida infection are pseudomembranous candidiasis (thrush), which may affect any of the mucosal surfaces, and acute erythematous candidiasis, a red and markedly painful variant commonly seen in AIDS patients.

In most cases, Candida infection may be controlled with antifungal medications used locally or systemically. Control is difficult, however, in patients with immune dysfunction, as in AIDS, or other chronic debilitating diseases. Often the organisms become resistant to standard therapy, and aggressive approaches are necessary (Tyldesley and Field, supra). The spread of oral candidiasis to the esophagus or lungs may be life-threatening and is one of the criteria used to define frank AIDS (Samaranayake and Holmstrup, J. Oral Pathol. Medi. 18: 554-64 (1989)).

Herpes Simplex Virus Infections

In any given year, about one-half-million Americans will experience their first encounter with the herpes simplex virus type 1 (HSV-1), the cause of cold sores. That first encounter usually occurs in the oral region and can be so mild as to go unnoticed. But in some people, particularly young children and young adults, infection may take the form of primary herpetic stomatitis, with symptoms of malaise, muscle aches, sore throat, and enlarged and tender lymph nodes, prior to the appearance of the familiar cold sore blisters. These blisters usually show up on the lips, but any of the mucosal surfaces may be affected. Bright-red ulcerated areas and marked gingivitis can also be seen (Tyldesley and Field, supra).

Herpes viruses also cause genital infections, which are transmitted sexually. Both HSV-1 and HSV-2 have been found in oral and genital infections, with HSV-1 predominating in oral areas and HSV-2 in genital areas (Wheeler, J. Am. Acad. Dermatol 18 (1 Pt 2): 163-8 (1988)). Herpes viruses have also been implicated as cofactors in the development of oral cancers. Crowded living conditions may result in greater contact with infected individuals, which aids in transmission of HSV (Whitley, Pathol. Biol 40(7): 729-34 (1992)).

Oral Human Papillomavirus Infections

There are more than 100 recognized strains of oral human papillomavirus (HPV), a member of the papovavirus family, implicated in a variety of oral lesions (Regezi and Sciubba, Oral pathology. Clinical-pathologic correlations, 2^{nd} ed., (1993)). Most common are papillomas (warts) found on or around the lips and in the mouth. HPV is found in 80 percent of these oral squamous papillomas (de Villiers, Biomed. Pharmacother. 43: 31-6 (1989)). The virus has also been identified in 30 to 40 percent of oral squamous cell carcinomas (Chang et al., Arch. Dermatol. Res. 282(8): 493-7 (1990)) and has been implicated in cervical cancer as well. Whether a cancer or nonmalignant wart develops may depend on which virus is present or on which viral genes are activated. Oral warts are most often found in children, probably as a result of chewing warts on the hands. In adults, sexual transmission from the anogenital region may occur (Franchesi et al., Cancer Epidemiol. Biomarkers Prev. 5: 565-75 (1996)). In general, viral warts spontaneously regress after 1 or 2 years. The immune system normally keeps HPV infections under control, as evidenced by the increased prevalence of HPV-associated lesions in HIV-infected patients and others with immunodeficiency.

Recurrent Aphthous Ulcers

Recurrent aphthous ulcers (RAU), also referred to as recurrent aphthous stomatitis, is the technical term for canker sores, the most common and generally mild oral mucosal disease. Between five and twenty-five percent of the general population is affected, with higher numbers in selected groups, such as health professional students (Ferguson et al, J. Oral Med. 39(4): 212-7 (1984); Kleinman et al., Community Dent. Oral Epidemiol. 5: 140-4 (1991)).

The disease takes three clinical forms: RAU minor, RAU major, and herpetiform RAU. The minor form accounts for 70 to 87 percent of cases. The sores are small, discrete, shallow ulcers surrounded by a red halo appearing at the front of the mouth or the tongue. The ulcers, which usually last up to two weeks, are painful and can make eating or speaking difficult. About half of RAU patients experience recurrences every one to three months; as many as thirty percent report continuous recurrences (Bagan et al., J. Oral Pathol. Med. 20: 395-7 (1991)).

RAU major accounts for seven to twenty percent of cases and usually appears as one to ten larger coalescent ulcers at a time, which may persist for weeks or months (Bagan et al., supra). Herpetiform RAU has been reported as occurring in seven to ten percent of RAU cases. The ulcers appear in crops of ten to one hundred at a time, concentrating in the back of the mouth and lasting for seven to fourteen days (Bagan et al., supra).

RAU can begin in childhood, but the peak period for onset is the second decade (Lehner, Proc. R. Soc. Med. 61: 515-24 (1968)). About fifty percent of close relatives of patients with RAU also have the condition (Ship, J. Dent. Res. 44: 837-44 (1965)), and a high correlation of RAU has been noted in identical but not fraternal twins. Associations have been found between RAU and specific genetic markers (Scully and Porter, J. Oral Pathol. Med. 18: 21-7 (1989)).

RAU has also been associated with hypersensitivities to some foods, food dyes, and food preservatives (Woo and Sonis, J. Am. Dent. Assoc. 127(8): 1202-13 (1996)). Nutritional deficiencies—especially in iron, folic acid, various B vitamins, or combinations thereof—have also been reported, and improvements noted with suitable dietary supplements (Nolan et al., J. Oral Pathol. Med. 20: 389-91 (1991)).

D. Oral and Pharyngeal Cancers and Precancerous Lesions

Oral cancer is the sixth most common cancer in U.S. males and takes a disproportionate toll on minorities; it now ranks as the fourth most common cancer among African American men (Kosary et al., SEER Cancer Statistics Review., NIH Pub. No. 96-2789 (1995)). The most common oral sites are on the tongue, the lips, and the floor of the mouth.

Viruses that have been implicated in oral cancer include herpes simplex type 1 and human papillomavirus. Epstein-Barr virus, also a herpes virus, is now accepted as an oncogenic virus responsible for Burkitt's lymphoma, occurring primarily in Africa, and nasopharyngeal carcinoma, occurring primarily in China. HPV is a major etiologic agent in cervical cancer, and has been found in association with oral cancer as well (Sugerman and Shillitoe, Oral Dis. 3: 130-47 (1997)). HPV DNA sequences have been found in oral precancerous lesions as well as in squamous cell carcinomas (Syrjanen et al., J. Oral Pathol. 17(6): 273-8 (1988)), and experimental evidence has shown that HPV-16 may be an important cofactor in oral carcinogenesis (Park et al., Oncogene 10(11: 2145-53 (1995)). Herpes simplex type 1 antibodies were demonstrated in patients with oral cancer, and herpes was found to induce dysplasia (abnormal cellular changes) in the lips of hamsters when combined with the application of tobacco tar condensate.

More recently, human herpes virus 8, a newly identified member of the herpes virus family, has been found in Kaposi's sarcoma, an otherwise rare cancer occurring in patients with AIDS. These tumors often appear initially within the oral cavity. (Epstein and Scully, Int. J. Oral Maxillofac. Surg. 21(4): 219-26 (1992)). Other uncommon oral malignant tumors, such as Hodgkin's lymphoma and non-Hodgkin's lymphoma, may also occur in the mouths of AIDS patients. In addition to viruses, infection with strains of the fungus Candida albicans has been linked to the development of oral cancers via the fungal production of nitrosamines, which are known carcinogens.

E. Associated Autoimmune Disorders

Oral, dental, or craniofacial signs and symptoms play a critical role in autoimmune disorders such as Sjögren's syndrome, and in a number of chronic and disabling pain conditions. Sjögren's syndrome is one of several autoimmune disorders in which the body's own cells and tissues are mistakenly targeted for destruction by the immune system. Like other autoimmune conditions, Sjögren's syndrome is more prevalent among women. The ratio of females to males affected is 9:1, with symptoms usually developing in middle age. There are an estimated one to two million individuals in the United States with Sjögren's syndrome (Talal, Rheum. Dis. Clin. North Am. 18(3): 507-15 (1992)).

The disease occurs in two forms. Primary Sjögren's involves the salivary and lacrimal (tear) glands. In secondary Sjögren's the glandular involvement is accompanied by the development of a connective tissue or collagen disease, most often rheumatoid arthritis, lupus erythematosis, scleroderma, or biliary cirrhosis.

The glandular involvement causes a marked reduction in fluid secretion, resulting in xerostomia and xerophthalmia (dry eyes). The constant oral dryness causes difficulty in speaking, chewing, and swallowing; the dry eyes often itch and feel gritty. There is no cure for Sjögren's, and patients often carry eye drops and water bottles or saliva substitutes in an attempt to provide symptomatic relief. Clinically, the reduction in salivary flow changes the bacterial flora, which, in addition to the reduction in salivary protective components, increases the risk of caries and candidiasis (Daniels and Fox, Rheum. Dis. Clin. North Am. 18: 571-89 (1992)). Recent studies have indicated that there is a reduction in masticatory function (Dusek et al. Gerodontology 13(1): 3-6 (1996)) and an increased prevalence of periodontal disease (Najera et al., Oral Surg. Oral Med. Oral Pathol Oral Radiol. Endod. 83(4): 453-7 (1997)). In advanced stages the salivary glands can swell because of obstruction and infection or lymphatic infiltration. In both forms of the disease, other systems can eventually become affected. Nasal, laryngeal, and vaginal dryness can occur, as well as abnormalities in internal organs (Oxholm and Asmussen, J. Intern. Med. 239: 467-74 (1996)). Patients with Sjögren's syndrome are at some risk of developing diseases such as non-Hodgkin's lymphoma; clinical data indicate that such lymphomas develop in 5 percent of patients with Sjögren's syndrome (Moutsopoulos et al., Am. J. Med. 64(5): 732-41 (1978)).

F. HIV and Osteoporosis

The mouth may serve as an early warning system, diagnostic of systemic infectious disease and predictive of its progression, such as with HIV infection. In the case where oral cells and tissues have counterparts in other parts of the body, oral changes may indicate a common pathological process. During routine oral examinations and perhaps in future screening tests, radiographic or magnetic resonance imaging of oral bone may be diagnostic of early osteoporotic changes in the skeleton.

HIV Infection

The progressive destruction of the body's immune system by HIV leads to a number of oral lesions, such as oral candidiasis and oral hairy leukoplakia, that have been used not only in diagnosis but also in determining specific stages of HIV infection. Oral candidiasis is rarely seen in previously healthy young adults who have not received prior medical therapy such as cancer chemotherapy or treatment with other immunosuppressive drugs. Oral candidiasis may be the first sign of HIV infection and often occurs as part of the initial phase of infection—the acute HIV syndrome (Tindall et al., “Primary HIV infection: Clinical, Immunologic, and Serologic Aspects,” in The Medical Management of AIDS, Sande et al., eds., pp. 105-129; W. B. Saunders, 1995). It tends to increase in prevalence with progression of HIV infection when CD4 lymphocyte counts fall. It also appears to be the most common oral manifestation in pediatric HIV infection (Kline, Pediatrics 97(3): 380-388 (1996) and has been demonstrated to proceed to esophageal candidiasis, a sign of overt AIDS. (Saah et al., Am. J. Epidemiol. 135: 1147-1155 (1992)). Both the pseudomembranous and the erythematous forms of candidiasis appear to be important predictors of progression of HIV infection (Klein et al., AIDS 6(3): 332-333 (1992)).

Like oral candidiasis, oral hairy leukoplakia in HIV-positive persons heralds more rapid progression to AIDS. Oral hairy leukoplakia is an oral lesion first reported in the early days of the AIDS epidemic. Since its discovery, hairy leukoplakia has been found in HIV-negative persons with other forms of immunosuppression, such as organ or bone marrow recipients and those on long-term steroid therapy, and less frequently among immunocompetent persons.

Linear gingival erythema and necrotizing ulcerative periodontitis may be predictive of progression of HIV infection. (Mealey, Ann. Periodontaol. 1: 256-321 (1996)). Necrotizing ulcerative periodontitis, a more serious periodontal condition observed in HIV-infected persons, is a good predictor of CD4+ cell counts of under 200 per cubic millimeter. In addition, the numerous ulcerative and nonulcerative conditions that affect the oral cavity may affect the biologic activity of HIV and are affected by its treatments. (Mealey, supra).

Osteoporosis and Oral Bone Loss

Osteoporosis, a degenerative disease characterized by the loss of bone mineral and associated structural changes, has long been suspected as a risk factor for oral bone loss. In addition, measures of oral bone loss have been proposed as potential screening tests for osteoporosis. Osteoporosis affects over 20 million people in the United States, most of whom are women, and results in nearly 2 million fractures per year. (National Institute of Arthritis, Musculoskeletal and Skin Diseases 2000). The disease is more prevalent in white and Asian American women than in black women. Oral bone loss has been reported to be more prevalent in women than in men. Also, the association between estrogen status, alveolar bone density, and history of periodontitis in postmenopausal women has been studied (Payne et al., J. Periodontol 6: 24-31 (1997)).

Larger cross-sectional studies, as well as longitudinal and mechanism studies, are needed to better define the relationship between osteoporosis, osteopenia, and oral bone loss, periodontal disease, and tooth loss. The role of factors involved in the regulation of bone mineral density in men as well as in postmenopausal women needs to be evaluated further with reference to oral bone loss, tooth loss, and periodontal disease. Variables such as sex, race, dietary calcium and phosphorus, vitamin D intake, exercise, body mass index, smoking, genetics, medication use, reproductive history, and psychosocial factors need to be assessed in depth. In addition, reliable and valid criteria and imaging technologies for assessing osteoporosis and oral bone loss are needed to better elucidate the full relationship between skeletal and mandibular bone mineral density, periodontal disease, alveolar ridge resorption, and tooth loss.

II. Salivary Analysis for Predicting Diseases

The present invention provides potent new saliva-based methodologies and technologies for predicting the risk of and treating a disease. Specifically, the present invention relates to compositions and methods for predicting the risk of a disease based on an anlysis of salivary mucins. For example, the invention provides compositions and methods for predicting the risk of disease based on quantitating sialic acid concentratin of MUC7 mucin. It is contemplated that other properties of MUC7 mucin and other salivary mucins will be used for predicting the risk of oral diseases and associated medical conditions. Thus, it is contemplated that the risk of oral diseases and associated diseases can be predicted by quantitating any saliva factor that demonstrates significant variation between subjects in a nested ANOVA.

A. Salivary Mucins

The functional properties of saliva proteins, known as salivary mucins, relative to oral health status are the subject of continuing research. (Ayad et al., J. Dent. Res. 79: 976-982 (2000)). The existence of high-molecular-weight glycoproteins in saliva and saliva secretions, called mucins, has been recognized for nearly thirty years. (Offner et al., “Heterogeneity of High-molecular weight Human Salivary Mucins,” Adv. Dent. Res. 14: 69-75 (2000)). Mucins are essential for oral health and perform many diverse functions in the oral cavity. For example, mucins are the principal protein components of the mucous layer which coats epithelial surfaces in the gastrointestinal, respiratory, and reproductive tracts. This layer forms a viscous barrier which protects the underlying epithelium from desiccation, mechanical injury, and microbial assault, while allowing for active absorption and secretion by mucosal cells. Mucins are also secreted by salivary glands and are thought to have a major role in the protection of oral epithelial surfaces, as well as in the non-immune host defense system in the oral cavity. (Offner et al., supra).

From a biochemical standpoint, mucins are comprised of approximately 15%-20% protein, and up to 80% carbohydrate, present largely in the form of O-linked glycans. (Strous and Dekker, “Mucin-like glycoproteins,” Crit. Rev. Biochem. Mol. Biol. 27: 57-92 (1992); Gendler and Spicer, “Epithelial mucin genes,” Ann. Rev. Physiol. 57: 607-634 (1995)). Serine and threonine are the most abundant amino acids and serve as the attachment sites for these carbohydrate chains. Many mucins have monomeric molecular weights greater than two million Daltons, and form multimers more than ten times that size. (Offner et al., supra). To date, eleven distinct human mucin genes have been isolated and have been numbered MUC1-MUC4, MUC5AC, MUC5B, MUC6-8, and MUC11-MUC12, in the order of their discovery.

These mucins share several common properties. The polypeptide backbone can be divided into three regions. The central region is enriched in serine, threonine, and sometimes proline, and contains tandemly repeated sequences ranging in length from 8 to 169 amino acids. This domain serves as the attachment site for the O-glycans, and each mucin has a unique, signature tandem-repeat sequence. The N- and C-terminal regions of mucins are non- or sparsely glycosylated with both O- and N-linked sugars. In many mucins, these flanking regions are cysteine-rich, containing nearly 10% cysteine. Mucins could be organized into three distinct classes: the large gel-forming mucins (i.e., MUC2, MUC5AC, MUC5B, and MUC6); the large membrane-associated mucins (i.e., MUC1, MUC3, MUC4, and MUC12); and the small soluble mucins represented by MUC7. Insufficient information is available to assign MUC8 and MUC11 to one of these categories. (Ofnner et al., supra).

The MUC7 gene has previously been reported. (Bobek et al., Genomics 31: 277-282 (1998)). The MUC7 mucin is generally regarded as having the ability to bind to and aggregate several species of oral bacteria, including several strains of S. mutans, and A. actinomycetemcomitans. The former is thought to be the most cariogenic of the oral bacteria and the latter is one of two major pathogens in periodontal disease. The MUC7 mucin also binds C. albicans and can have candidicidal activity. Desialylation of the mucin apparently destroys its ability to aggregate some species of oral bacteria. Recent studies further indicate that MUC7 mucin binds oral neutrophils on a different oligosaccharide motif than is used to bind oral bacteria. Since oral neutrophils can phagocytize oral bacteria, perhaps this dual property facilitates opsinization. If true, this property alone could have a major impact on the bacterial count in the oral cavity. With regard to the primary site of binding to oral bacteria, recent studies suggest that a non-glycosylated domain of MUC7 mucin can be more responsible than its oliogosaccharides.

According to the invention, various characteristics of salivary mucins can be associated with disease states. For example, MUC7 mucin characteristics such as the total apomucin (MUC7 mucin without its attendent carbohydrates), carbohydrate, and sialic acid associated with MUC7 mucin in saliva can be separated and individually quantitated for predicting the risk of disease. Thus, according to one embodiment of the invention, the risk of oral diseases and associated diseases is predicted by quantitating the total carbohydrate content, including the number and types of carbohydrate chains on the MUC7 mucin from unstimulated or stimulated saliva. Similarly, in another embodiment, the risk of oral diseases and associated diseases is predicted by quantitating the total apoprotein content of the MUC7 mucin from unstimulated saliva.

It is expected that polymorphism will lead to variations in the characteristics of salivary mucins from subject to subject. For example, it is known that different subjects have different classes of oligosaccharides on MUC7 mucin (Prakobphol et al., Biochemistry 38: 6817-6825 (1999)). Therefore, according to the invention, an subject's genotype is associated with the characteristics of the subject's salivary mucins. Conversely, the invention provides for predicting the characteristics of an subject's salivary mucin by determining the subject's genotype at a mucin genetic locus. Thus, for example, the genotype at the MUC7 genetic locus can be associated with the sialic acid content of the MUC7 mucin. This, then, allows the prediction of the sialic acid content of a subject's MUC7 mucin based on a determination of the subject's genotype at the MUC7 genetic locus. Accordingly, the invention provides for methods and compositions for determining the genotype of a subject at a mucin genetic locus.

B. Sampling Methods

In a preferred embodiment, the methods of the present invention analyze an unstimulated or stimulated saliva sample to test for the risk of a disease. Saliva specimens for testing can be collected following various methods known in the art. Proper conditions for generating unstimulated saliva have been described. (Nazaresh and Christiansen, J. Dent. Res. 61: 1158-1162 (1982)). Methods and devices for collecting saliva have also been described. (See also, U.S. Pat. No. 5,910,122 to D'Angelo; U.S. Pat. No. 5,714,341 to Thieme et al.; U.S. Pat. Nos. 5,335,673 and 5,103,836 to Goldstein et al.; U.S. Pat. No. 5,268,148 to Seymour; and U.S. Pat. No. 4,768,238 to Kleinberg et al., incorporated herein in their entirety by reference). It is contemplated that the methods of the present invention can also be practiced by analyzing stimulated saliva.

Furthermore, the methods of the present invention are not limited to performing salivary analysis immediately after collection of the sample. In other embodiments, salivary analysis following the methods of the present invention can be performed on a stored saliva sample. The saliva sample for testing can be preserved using methods and apparatuses known in the art. (See e.g., U.S. Pat. No. 5,968,746 to Schneider, hereby incorporated in its entirety by reference).

It is also contemplated that the methods of the present invention be used to perform salivary analysis on saliva samples that have been treated to reduce its viscosity. Mucopolysaccharide-containing body fluids, such as saliva, contain antibodies and other metabolites that are useful in the diagnosis of diseases, including those of bacterial, viral, and metabolic origin. However, the viscous nature of such fluids, due to the nature of mucopolysaccharides, makes testing of these fluids difficult. In order to prepare saliva for any laboratory testing procedure, the saliva must be rendered sufficiently fluid (i.e., viscosity must be reduced) and free from debris. Techniques used to remove debris include centrifugation and filtration. The viscosity of saliva can also be reduced by mixing a saliva sample with a cationic quaternary ammonium reagent. (See, U.S. Pat. No. 5,112,758 to Fellman et al., incorporated herein in its entirety by reference).

Further, it is contemplated that the methods of the present invention be used in analyzing factors from saliva samples obtained from a subject suffering from xerostomia. Xerostomia is a condition in which the salivary glands do not produce sufficient quantities of saliva. The onset of the effects of xerostomia is insidious, with no clear line of demarcation when one suffers from the malady. It is estimated that several million individuals suffer from this condition nationwide. The actual number of individuals suffering from xerostomia is not known, however, because there has been little acknowledgement of the prevalence or severity of the problem until recently. It is estimated that about ten percent of the population over 50 years of age and 25 percent of the population over 65 years of age suffer from xerostomia. The majority of those affected are women.

Some direct primary causes of xerostomia are autoimmune diseases, such as Sjögren's syndrome, medical irradiation, malnutrition, hormonal imbalance, arthritis and aging. When areas of the head or neck are medically irradiated by as little as 1000 rads per week, 85 percent of the patients suffer from xerostomia after six weeks and 95 percent after three months. Radiation xerostomia onsets rapidly with a greater than 50 percent decrease in salivary flow after one week, and a greater than 75 percent decline after six weeks of treatment. The xerostomia is progressive, persistent, and irreversible, reaching a greater than 95% reduction in saliva output three years after radiation. In patients where only part of the major salivary glands is in the path of the ionizing radiation, the non-exposed portion can undergo hyperplasia and partly compensate for the damaged acini. The most severe cases of xerostomia are caused by radiation therapy after head and neck surgery, and by autoimmune diseases such as lupus, Sjögrens Syndrome, and rheumatoid arthritis. See e.g., P. C. Fox et al., J. Am. Dental Assoc. 110:519-525 (1985). Secondarily, xerostomia is a side effect from the administration of over 400 drugs, including major antihypertensives, antidepressants, antispasmodics, diuretics, muscle relaxants, antipsychotics, appetite depressants, and therapeutics for Parkinson's disease.

To predict the risk of a disease in a subject suffering from xerostomia, it is contemplated that various methods for enhancing saliva be used to obtain a salivary sample for analysis. Various methods for enhancing saliva are known in the art. For example, U.S. Pat. No. 5,886,054 (incorporated herein in its entirety by reference) teaches a therapeutic method for enhancing saliva, using an aqueous solution of at least one polymer and one electrolyte. The aqueous solution is preferably buffered and optionally contains at least one mucin. In another example, U.S. Pat. No. 6,230,052 (incorporated herein in its entirety by reference) teaches an implantable device for inducing salivation by neural stimulation at neurally sensitive location within an oral or perioral tissue of a user.

It is understood that the examples for sampling saliva described above are for illustrative purposes only. It is also understood that various modifications for sampling saliva are contemplated to be within the scope of the present invention.

C. Analytical Methods

In one embodiment, the methods of the present invention comprise the step of separating a salivary mucin, e.g., MUC7 mucin (MG2), from all other sialic acid-containing molecules in the saliva. The sialic acid attached to the mucin is then quantitated and reported. For example, in a test for quantitating sialic acid associated with MUC7 mucin in saliva developed by Denny et al. (Denny et al., J. Dent. Res. 70: 1320-1327 (1991)), MUC7 mucin is separated from other sialic acid-containing components using sodium dodecylsulfate-polyacrylamide gel electrophoresis (SDS-PAGE). The resulting gel is then stained with a dye that binds only to the sialic acid of MUC7 mucin. (Baughan et al., Oral Microbiol. Immunol. 15: 10-14 (2000)). Finally, the amount of staining of the MUC7 mucin band on the gel is quantitated against a known MUC7 mucin standard, and then reported as units per milliliter of the original saliva sample.

It is not intended that the present invention be limited to specific methods of quantitating and reporting the sialic acid concentration of a salivary mucin. The sialic acid associated with a mucin can be quantitated by any of a variety of approaches well-known in the art, such as dye-binding, lectin affinity, or chemical reaction.

It is also not intended that the present invention be limited to specific methods of separating sialic acid associated with mucin in saliva. Various tests depending upon the proportional, selective binding of a mucin to surfaces, either by direct interactions or by facilitated binding, are contemplated by the present invention. Any substrate that selectively binds to a mucin, such as MUC7 mucin, can be used to practice the compositions and methods of the present invention. For example, the compositions and methods of the present invention can involve facilitated binding, which initially involves linking a specific binding pair (e.g., an antibody or lectin) to the surface. Compositions and methods for linking and detecting immunological binding pairs (e.g., antigen-antibody) are well-known in the art. (See e.g., U.S. Pat. No. 5,447,837 to Urnovitz; U.S. Pat. No. 5,741,662 to Madsen et al.; U.S. Pat. No. 4,962,023 to Todd et al.; incorporated herein in their entirety by reference). Compositions and methods for linking and detecting non-immunological binding pairs are also well-known in the art. (See e.g., U.S. Pat. No. 5,374,516 to Sutton et al., incorporated herein in its entirety by reference).

It is also contemplated that the constituents of saliva samples be analyzed using capillary electrophoresis. Capillary electrophoresis is a highly efficient method for the separation and detection of molecules. Conventional methods of electrophoresis are limited by the heat induced during a run. Capillary tubes, in contrast, can be run at very high voltage gradients, due to their excellent heat transfer ability. The capillary tubes used are hollow silica glass with polyimide coating on the exterior to prevent breakage. The silica wall gives a net negative charge to the inner surface. The action of an electric field on positive counterions next to the negatively charged inner wall causes the bulk flow of liquid known as electro-osmotic flow (“EOF”). Separation of molecules is a combined result of the effects of EOF and preferential electrophoretic mobility.

Free solution capillary electrophoresis runs the sample into a single, continuous buffer. Electrolyte buffers can be simple salts, such as borate and phosphate, or can contain additives. Micellar electrokinetic capillary chromatography adds detergents above their critical micellar concentration, thereby allowing the separation of neutral molecules on the basis of hydrophobicity. Other available methods are adapted from slab gel electrophoresis, such as isoelectric focusing, which resolves the samples by isoelectric point, or the addition of linear or cross-linked polymer to allow molecular sieving to take place. When clathrates are added, stereoisomers can be separated.

A major advantage of capillary electrophoresis is the speed at which components can be capable of being resolved, coupled with reproducibility and high level of sensitivity. It is therefore desirable to provide methods for clinical sample analysis which can take advantage of these properties. (See e.g., U.S. Pat. No. 5,536,382 to Sunzeri, incorporated herein in its entirety by reference).

D. Statistical Tests

The statistical tests involved in the discovery of the predictive nature of the combination of unstimulated or stimulated saliva, and the test for mucin-associated sialic acid are standard and well-known in the art of statistical analyses. Specifically, the present invention used Pearson's correlation coefficient, simple linear regression analysis, multiple linear regression analysis, and ANOVA. An aspect of the reliability for the prediction of dental caries risk is the accuracy of representation involving the relationship between the test results and the observed decay or fillings on teeth (DFT) in the standard population. In most statistical programs, this relationship is calculated by the least squares method, and yields the mathematical formula from which the linear regression line is derived and predictions can be made. However, the typical regression line takes into account only the variation of the dependent variable. In a particular embodiment, the variation of dental caries experience is a dependent variable.

In an embodiment of the invention where there is a normal variation in DFT and MUC7 mucin concentration, a different type of statistical test can be used to give the most representative mathematical regression equation. This approach to regression analysis can be performed by a variety of statistical tests, such as orthogonal least squares, geometric mean regression, Bartlett's, three-group method (i.e., for Type II regression analysis), and random variable regression analysis. These alternative methods can also be used to calculate the mathematical description of the regression line on the data. In this embodiment, these methods did not measurably alter the predictive outcomes obtained by traditional simple linear regression analysis.

E. Test Versions

The present invention also provides compositions for diagnosing diseases. Specifically, the present invention provides a composition for diagnosing a disease, comprising: i) a means for collecting saliva; ii) a means for isolating the mucin from other sialic acid-containing molecules in the saliva, to produce an isolated mucin; iii) a means for measuring the amount of sialic acid associated with the isolated mucin; and iv) an oral fluid standard for evaluating the amount of sialic acid in the isolated mucin. In one embodiment, the mucin is MUC7 mucin. In another embodiment, the mucin is MUC5 mucin. The saliva is either unstimulated or stimulated saliva. Different versions of the compositions and methods of the present invention can be used for various applications.

For example, one test version could be used in a non-clinical setting to provide a general forecast of cumulative caries experience, as well as to assess the risk of future caries development (e.g., high, medium or low risk for future caries development). This version would also be appropriate for use in underdeveloped regions, so that limited oral health resources can O be targeted at those who are deemed most in need of care, thereby supporting cost-effective community-based health programs.

Another test version could be used to quantitate dental caries risk leading to the prediction of future caries experience at subsequent ages. This test might be administered in a dentist's office where appropriate countermeasures could be initiated. Yet another test version would be diagnostic and used with medically compromised patients, such as those suffering from diabetes or AIDS. Still another test version would feature multiple sample, high throughput characteristics. The use of this test version would be targeted to screening populations of saliva samples, such as those used for epidemiological surveys.

Yet another test version can be computer-based. For example, the computer-based system for predicting future health described in U.S. Pat. No. 6,059,724 to Campbell et al. (incorporated herein in its entirety by reference) can be used to practice the methods of the present invention. Specifically, the computer-based system can comprise: (a) a computer comprising a processor containing a database of longitudinally-acquired biomarker values from individual members of a test population and subpopulations; and (b) a computer program that includes steps for: (1) selecting from the biomarkers a subset of biomarkers for discriminating between members belonging to the subpopulations; and (2) using the distributions of the selected biomarkers to develop a statistical procedure that is capable of being used for: (i) classifying members of the test population as belonging within a subpopulation having a prescribed high probability of acquiring the specified biological condition; or (ii) estimating quantitatively, for each member of the test population, the probability of acquiring the specified biological condition within the specified time period or age interval.

III. Methods for Preventing Diseases

The present invention also provides methods for preventing diseases. In particular, the compositions and methods of the present invention can be used for preventing oral diseases and associated diseases. Once symptoms of associated diseases (e.g., cardiovascular and respiratory diseases) are detected, treatment is difficult and expensive. Thus, treatment results would be much better if individuals could be determined to be at risk prior to symptoms. In this manner, preventive measures could be taken and early intervention strategies could be employed.

In one embodiment, the present invention provides a method for preventing the risk of a disease in a subject, comprising the steps of: a) providing a saliva sample from a subject; b) isolating the mucin from the saliva sample; c) quantitating the sialic acid content of the isolated mucin; and d) administering an anti-caries reagent when the sialic acid content in the isolated mucin falls significantly below the level expressed in a normal control (i.e., a subject free from the disease being tested for. In one embodiment, the mucin is MUC7 mucin. In another embodiment, the mucin is MUC5 mucin. The saliva is either unstimulated or stimulated saliva. In some embodiments, the normal control comprises an oral fluid standard.

A. Oral Fluid Standards

Various oral fluid standards for testing, calibration and standardization of devices and methods for the analysis of oral fluids are well-known in the art. (See e.g., U.S. Pat. Nos. 5,736,322 and 5,695,929 to Goldstein, incorporated herein in their entirety by reference). U.S. Pat. No. 5,736,322 describes oral fluid standards composed of an aqueous solution of a mucin and a protease inhibitor. A preferred oral fluid standards additionally includes an amylase. Any protease inhibitor that reduces or eliminates proteolytic activity associated with a mucin is suitable. Preferred protease inhibitors inhibit the papain-like (cysteine) proteases. Particularly preferred protease inhibitors include, but are not limited to, leupeptin, antipain, benzamidine, chymostatin, pepstatin A, and aprotinin. In a particularly preferred embodiment, the mucin is present at a concentration ranging from about 0.001% to about 0.4% (w/v); the amylase is present at a concentration ranging from about 0.1 g/L to about 5.0 g/L; and the protease inhibitor is present in a concentration sufficient to reduce or prevent proteolysis of antibodies added to the oral fluid standard.

The oral fluid standards can additionally include one or more components selected from the group consisting of magnesium, calcium, sodium, phosphate, chloride, potassium, and bicarbonate. The oral fluid standard can additionally include a preservative, most preferably a preservative selected from the group consisting of thimerosal, gentamycin, chlorhexidine digluconate, and polyhexamethylenediguanide.

The standard oral fluid standard can include serum, more preferably human serum. The serum can be positive or negative for an analyte including, but not limited to any of the above-identified analytes. A particularly preferred oral fluid standard includes nitrite at a concentration ranging from about 0.1 mM to about 2 mM; magnesium at a concentration ranging from about 0.03 mM to about 0.6 mM; calcium at a concentration ranging from about 0.5 mM to about 5.0 mM; sodium at a concentration ranging from about 2 mM to about 80 mM; phosphate at a concentration ranging from about 1.8 mM to about 25 mM; chloride at a concentration ranging from about 10 mM to about 56 mM; potassium at a concentration ranging from about 10 mM to about 40 mM; and bicarbonate at a concentration ranging from about 2 mM to about 35 mM. This standard can additionally include a preservative.

Similarly, the oral fluid standards can additionally include one or more analytes. Suitable analytes include, but are not limited to an antibody selected from the group consisting of an antibody to HIV-1, an antibody to HIV-2, an antibody to HTLV-1, an antibody to HTLV-2, an antibody to Helicobacter pylori, an antibody to hepatitis A, an antibody to hepatitis B, an antibody to hepatitis C, an antibody to measles, an antibody to mumps, an antibody to rubella, cotinine, cocaine, benzoylecgonine, benzodiazapine, tetrahydrocannabinol, theophylline, phenytoin, β-hCG, thyroxine, thyroid stimulating hormone, follicle stimulating hormone, luteinizing hormone, glucose, insulin, or cholesterol.

U.S. Pat. No. 5,696,929 to Goldstein also describes a saliva standard for measuring the efficacy of saliva collection kits and for comparing and standardizing analytical methods. Generally, the inventive substitute saliva standard has the composition:

| Ingredients | mmol/liter |
|---|---|
| Nitrite | 0.1-0.2 |
| Magnesium | 0.15-0.6 |
| Calcium | 0.5-0.47 |
| Sodium | 2-80 |
| Phosphate | 1.5-25 |
| Chloride | 10-56 |
| Potassium | 13-40 |
| Bicarbonate | 2-35 |
| Thimerosal | 0.01-0.1 g/100 ml |
| Amylase | 0.025-0.1 g/100 ml |
| Mucin (5%) | 0.02-0.5 g/liter |
| Antipain | 0.05 mg/liter |
| Deionized Water | QS to 1 L (approx. eq. 998 ml) |

In order to test a particular assay, a given amount of the substitute saliva standard is spiked with a predetermined amount of analyte, and the desired dilution made. The assay is then run. The substitute saliva standard could be spiked with, e.g., HIV antibody-positive serum, HIV antibody-negative serum, or any other target analyte which would ordinarily be detectable in human saliva. Representative of such analytes are those mentioned in the aforementioned U.S. Pat. No. 5,103,836 (incorporated herein in its entirety by reference).

B. Anti-Caries Reagents

It is contemplated that various anti-caries reagents well-known in the art can used to practice the methods of the present invention. For example, U.S. Pat. No. 6,136,298 to Gaffar et al. (incorporated herein in its entirety by reference) describes oral compositions containing a substantially water insoluble noncationic antimicrobial agent, such as triclosan or xylitol for inhibiting S. mutans and dental caries. Typical examples of water insoluble noncationic antibacterial agents which are particularly desirable from considerations of effectiveness, safety and formulation are: halogenated diphenyl ethers; benzoic esters; sesquiterpene alcohols such as farnesol, nerolidol, bisabolol, santalol and like compounds; halogenated carbanilides; and phenolic compounds (including phenol and its homologs; mono-, poly-alkyl and aromatic halo-phenols; resorcinol and catechol and their derivatives; and bisphenolic compounds. The noncationic antibacterial agent is present in the dentifrice in an effective antiplaque amount, typically about 0.01-5% by weight, preferably about 0.03-1.0% by weight and most preferably about 0.3-0.5% by weight. The antibacterial agent is substantially water-insoluble, meaning that its solubility is less than about 1% by weight in water at 25° C., and can be even less than about 0.1% by weight.

The preferred halogenated diphenyl ether and most preferred noncationic antibacterial agent is triclosan. Preferred other noncationic antibacterial agents are hexyl resorcinol and 2,2′-methylene bis (4-chloro-6-bromophenol). Xylitol, when present in amounts ranging from about 0.1% by weight to about 40% by weight, also enhances the antibacterial and anticaries properties of the oral compositions described above.

U.S. Pat. No. 5,807,541 to Aberg et al. (incorporated herein in its entirety by reference) describes compositions and methods for inhibiting the development of caries using non-steroidal anti-inflammatory drugs (NSAIDs) and fluoride reagents. NSAIDS can be characterized into five groups: (1) the propionic acids; (2) the acetic acids; (3) the fenamic acids; (4) the biphenylcarboxylic acids; and (5) the oxicams.

Propionic acid NSAIDs are non-narcotic analgesics/nonsteroidal antiinflammatory drugs having a free—CH(CH₃)COOH group, which optionally can be in the form of a pharmaceutically acceptable salt group, e.g.,—CH(CH₃)COO⁻Na⁺. The propionic acid side chain is typically attached directly or via a carbonyl function to a ring system, preferably to an aromatic ring system. Exemplary propionic acid NSAIDS include: ibuprofen, indoprofen, ketoprofen, naproxen, benoxaprofen, flurbiprofen, fenoprofen, fenbufen, pirprofen, carpofen, oxaprozin, pranoprofen, miroprofen, tioxaprofen, suprofen, alminoprofen, tiaprofen, fluprofen, and bucloxic acid. Structurally related propionic acid derivatives having similar analgesic and antiinflammatory properties are also intended to be included in this group.

Acetic acid NSAIDs are non-narcotic analgesics/nonsteroidal antiinflammatory drugs having a free—CH₂COOH group (which optionally can be in the form of a pharmaceutically acceptable salt group, e.g.—CH₂COO⁻Na⁺, typically attached directly to a ring system, preferably to an aromatic or heteroaromatic ring system. Exemplary acetic acid NSAIDS include: ketorolac, indomethacin, sulindac, tolmetin, zomepirac, diclofenac, fenclofenac, alclofenac, ibufenac, isoxepac, furofenac, tiopinac, zidometacin, acematacin, fentiazac, clidanac, oxpinac, and fenclozic acid. Structurally related acetic acid derivatives having similar analgesic and antiinflammatory properties are also intended to be encompassed by this group.

Fenamic acid NSAIDs are non-narcotic analgesics/nonsteroidal antiinflammatory drugs having a substituted N-phenylanthranilic acid structure. Exemplary fenamic acid derivatives include mefenamic acid, meclofenamic acid, flufenamic acid, niflumic acid, and tolfenamic acid. Biphenylcarboxylic acid NSAIDS are non-narcotic analgesics/nonsteroidal antiinflammatory drugs incorporating the basic structure of a biphenylcarboxylic acid. Exemplary biphenylcarboxylic acid NSAIDs include diflunisal and flufenisal. Oxicam NSAIDs” are N-aryl derivatives of 4-hydroxyl-1,2-benzothiazine 1,1-dioxide-3-carboxamide. Exemplary oxicam NSAIDs are piroxicam, sudoxicam and isoxicam.

Certain histidine-rich polypeptides (“HRPs,” also referred to as histatins) having a substantial proportion L-histidine (i.e., between about 14 and 40 mole and amino acid residues), have antibacterial and antifungal properties, particularly against S. mutans and Candida albicans. (U.S. Pat. No. 4,725,576 to Pollock et al.). HRPs are administrable to the loci of infection, particularly in the oral surfaces. Delivery can be by any conventional means, preferably topical means. In the case of oral administration, this would include dentifrices; mouthwashes; denture washes or soaks; denture adhesives or cements; and incorporation into polymers associated within the denture, particularly with the interface of the denture with the gum. Histatin-based peptides having antibacterial and antifungal properties are also described in U.S. Pat. Nos. 5,912,230; 5,885,965; 5,631,228; 5,646,119; and 5,486,503 to Oppenheim et al. (incorporated herein in their entirety).

U.S. Pat. No. 5,801,226 to Cummins et al. (incorporated herein in its entirety by reference) describes sodium- and stannous fluoride, aminefluorides, monosodiumfluoro-phosphate, casein, and plaque buffers such as urea, calcium lactate, calcium glycerophosphate, strontium polyacrylates, as anti-caries reagents.

U.S. Pat. No. 5,013,542 to Hay et al. (incorporated herein in its entirety by reference) describes compositions containing non-immunogenic amino acid segments of proline-rich proteins for inhibiting the adhesion of disease-causing microorganisms to tooth surfaces. Such microorganisms include, but are not limited to S. mutans, S. sanguis, S. sobrinus, Actinomyces viscosus, and Bacteroides gingivalis. The amino acid segment can be obtained from acidic, proline-rich proteins, such as those derived from human saliva. These proline-rich proteins show marked charge, structural asymmetry and exceptional reactivity to apatitic surfaces. When intact, these proline-rich proteins also promote the adhesion of microorganisms to apatitic surfaces. Because they are derived from human proline-rich proteins, they are recognized as “self” by humans, and antibodies to them have not been reported in humans. The mineral-binding segments can be used as the active ingredients alone or in combination with the other compounds, such as enzymes, antimicrobial agents, etc., in various compositions used for the treatment of the teeth so as to limit the adhesion and/or growth of microorganisms.

The active ingredient can be derived from segmenting a natural or synthetic, proline-rich protein, to provide a non-immunogenic ingredient. The non-immunogenic amino acid segment can be obtained by various techniques, such as by cloning, or by synthesizing analogs of the natural molecules or their segments by chemical means. The non-immunogenic amino acid segment can also be obtained enzymatically or by cleaving the proline-rich protein derived from human saliva by the enzyme trypsin. The removed portion of the proline-rich protein contains the bacterial binding sites. A variety of human, proline-rich phospho-proteins can be employed.

U.S. Pat. No. 6,231,857 to Shi et al., incorporated herein in its entirety be reference, describes antibodies of S. mutans, which can be used in treating dental caries. Specifically, Shi et al. describe three monoclonal IgG antibodies, each of which specifically binds an antigen on the surface of S. mutans. One monoclonal antibody is produced by a hybridoma deposited with the American Type Culture Collection as ATCC No. HB12559, and is designated SWLA1. A second monoclonal antibody is produced by a hybridoma deposited with the American Type Culture Collection as ATCC No. HB 12560, and is designated SWLA2. The third monoclonal antibody is produced by a hybridoma deposited with the American Type Culture Collection as ATCC No. HB 12258, and is designated SWLA3.

IV. Results

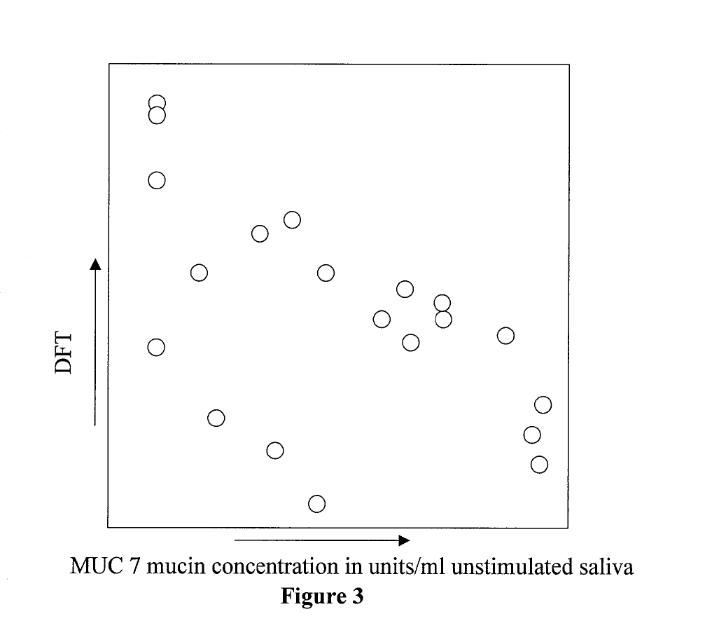

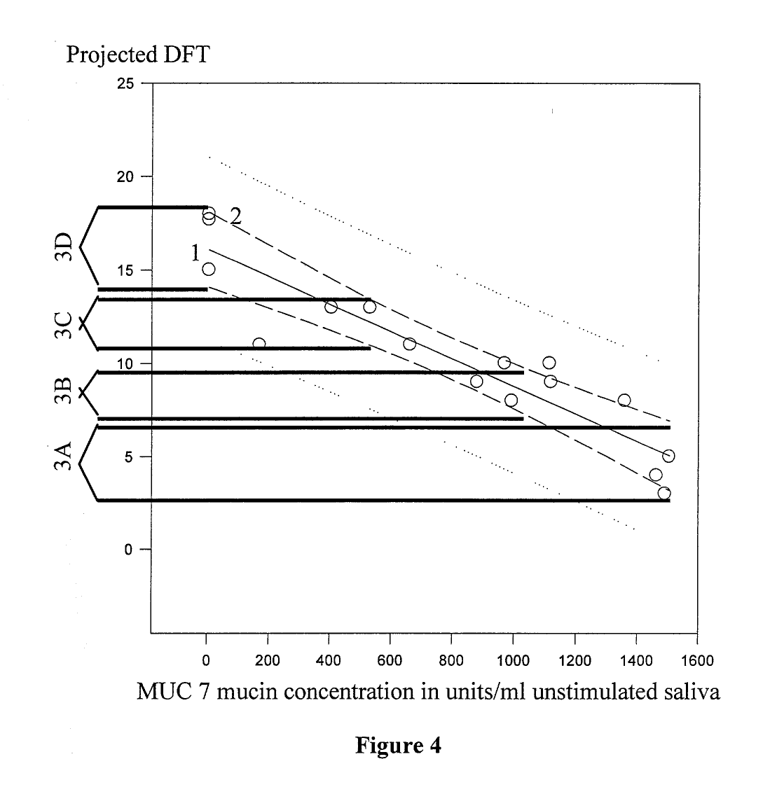

The methods of the present invention provide a correlation between the sialic acid concentration associated with the MUC7 mucin and the dental caries experience. The hallmark relation between DFT and MUC7 mucin in the 16 subject group minus the outlier group, has a correlation coefficient of −0.9252, and a highly significant p-value of 0.0000002866 (FIGS. 3 and 4). The adjusted r² (coefficient of determination) of this relationship is 0.8458, indicating that more than 84% of the variation in caries experience between subjects can be attributed to the results of their saliva tests (Table 1). Furthermore, if the age of the subject is included in the multiple linear regression analysis of the test data, the correlation coefficient increases to 0.957, and the adjusted coefficient of determination to 0.902 (Table 1). The straightforward prediction of dental caries risk at these levels of certainty, has not been possible with any other saliva test.

The MUC7 mucin (MG2) concentration in unstimulated saliva (uMUC7) of the group of 16 minus outliers (r²=0.846) is also a good predictor of the number of teeth with decay or fillings (DFT). There is also a significant predictor in the “total” group of 20 subjects (r²=0.275) (Table 1). Other significant but incremental contributers to the prediction of DFT are shown in Table 1. It is noteworthy that in the total group of 20 though the r² for MUC7 mucin concentration alone is relatively low, when it is combined with age and other factors the r² of the prediction is elevated to 0.782 (Table 1).

TABLE 1

| | DFT prediction characteristics | Total r² |
|---|---|---|
| Total group of 20 young | uMUC7 (27.5%) | r² = 0.782 |
| adults | Age (43.4%) | |
| | uMUC5B (4.7%) | |
| | sMUC5B (2.3%) | |
| | Stimulated Flow Rate (0.34%) | |
| Group of 16 young adults | uMUC7 (84.6%) | r² = 0.931 |
| minus outliers | Age (5.6%) | |
| | uMUC5B (2.8%) | |
| | sMUC5B (0.08%) | |

Terms;
uMUC7 = MUC7 mucin concentration in unstimulated saliva, uMUC5B = MUC5B mucin concentration in unstimulated saliva, sMUC5B = MUC5B mucin in stimulated saliva, Age = age of subject, Stimulated flow rate = chewing stimulated flow rate of saliva.

Table 2 illustrates that other factors can to varying degrees also forecast DFT, thus the intent is not to restrict this invention to MUC7 mucin concentration in unstimulated saliva. However, the strength of the relationship of MUC7 mucin concentration in unstimulated saliva to DFT provides the best illustration of the invention at this time.

TABLE 2

| Other Significant Predictors of DFT | r² |
|---|---|
| Age | r² = 0.598 in the total |
| | group and 0.562 for the |
| | group minus outliers |
| Stimulated MUC7 mucin (sMUC7) | r² = 0.056 [NS] for the total group |
| concentration | and 0.327 for group minus outliers |
| Unstimulated MUC7 mucin | r² probably similar to r² derived |
| (uMUC7) individual means | from single values of a large |
| | number of individuals |
| Unstimulated MUC5B mucin (MG1) | r² = 0.860 relative to |
| individual means | uMUC7 individual means |
| Gender difference in both DFT | Similar regression formulas for |
| and uMUC7 | both sexes (males having higher |
| | levels of uMUC7 and fewer DFT) |
| S. mutans titer in saliva | r² = 0.237 |

Terms:
Most are the same as in Table 1. Additionally “individual mean” refers to the average mucin concentration from 6 weekly collections from the same individual, and “S. mutans titer” is the S. mutans bacteria concentration in saliva of 24 older adults.

The data provides a regression line to predict DFT for subjects ages 18-35. However, it is not intended that the present invention be limited to predicting DFT for subjects aged 18-35. As would be obvious to one of ordinary skill in the art, the present invention can be used for predicting DFT, DMF and/or DMFS risk and experience for subjects within different age groups, using age appropriate regression lines. The present invention can also be used to predict general caries risk and experience as high, medium, and low, in subjects aged two through eighty and above.

The factor that has a significant degree of predictive impact for nearly every age group is past caries experience. (Powell, Community Dent. Oral Epidemiol. 26: 361-371 (1998); Lenander-Lumikari and Loimaranta, Adv. Dent. Res. 14: 40-47 (2000)). The caries experience of individuals generally progresses with age. A second predictor of caries risk is the titer of S. mutans in the oral cavity, although this predictor is consistently significant only within the first two years of age. The MUC7 mucin sialic acid concentration in unstimulated saliva has been shown to be inversely correlated with S. mutans titer in the saliva. (Baughan et al., Oral Microbiol. Immunol. 15: 10-14 (2000)). The MUC7 mucin sialic acid concentration in unstimulated saliva is inversely correlated with past (cumulative) caries experience. (Table 1) Thus, the present invention is a clear predictor of the two best recognized risk factor for dental caries, and is likely to be the single most important primary endogenous determinant of this risk.

V. Advantages of the Present Invention

The compositions and methods of the present invention present the following advantages. First, scientific evidence suggests that the MUC7 mucin concentration is likely to be one of the key determinants of the S. mutans titers in saliva. Thus, the present invention allows the prediction and diagnosis of the cariogenesis process at an earlier stage than S. mutans titer alone, and provides more avenues of prevention.

Second, the experimental results of the present invention show a clear numerical relationship to caries experience, in contrast to currently available technology for detecting S. mutans using DENTOCULT® Strip Mutans (“SM”) test strips (manufactured by Orion Diagnostica, Finland). At best, the DENTOCULT® SM test strips can differentiate the S. mutans titers in saliva into categories of high, medium, low and none. The present invention is also advantageous over DENTOCULT® SM strips because of the simplicity and ease of use. In a preferred embodiment, the methods of the present invention can be evaluated in a non-clinical setting by non-technical personnel. In contrast, the DENTOCULT® SM strips must be cultured under sterile conditions and evaluated by a trained, experienced personnel.

The present invention also provides non-invasive compositions and methods for predicting and diagnosing the risk of a disease in a subject. Numerous analytical methods have been developed for determining the presence or absence of, and/or quantifying the amount of various analytes in tissues and fluids of organisms. Currently most diagnostic testing is done with either blood, urine, fecal material, or tissue biopsy. Testing based on these materials, however, entails substantial invasion of privacy, and poses a significant safety hazard (particularly with testing of blood). In contrast, the collection of oral fluid for testing, including saliva and/or mucosal transudate, entails relatively little invasion of privacy, is relatively safe, and can be accomplished rapidly with relative ease.

In accordance with the present invention, saliva can replace blood in diagnostic procedures, the current standard for testing many diseases and conditions (e.g., diabetes, infectious disease, Parkinson's disease, alcoholic cirrhosis, Sjögren's syndrome, and cystic fibrosis sarcoidosis). With the methods and compositions of the present invention, new diagnostic tests for early disease detection, defining individual patient risk of adverse response to drugs, monitoring therapeutic progress, and determining outcomes of treatment are possible. The present invention provides saliva diagnostics that have selectivity, sensitivity, appropriate response time, dynamic range (values of interest), representative sampling, reliability or stability as well as the ability to assess multiple substances simultaneously.

### EXAMPLES

### Example 1

### Saliva Collection and Subject Selection

The test population comprises a typical young adult population (aged 18-35 years). All subjects agreed to participate voluntarily. No criteria precluded voluntary participation by this age range from the study group except that participants should have been deemed healthy, as determined from a questionnaire. This questionnaire focused mainly on use of medication, which may have the potential to impact or alter either saliva flow or composition, or on chronic health conditions, that might potentially affect gastrointestinal tract or respiratory tract secretions. The results showed that there appears to be a subpopulation of four males (18-22 years old) whose actual DFT is substantially lower than the value predicted from the MUC7 mucin data. However, there was no intent a priori to exclude or create subpopulations.

A questionnaire was given to all volunteers who were then asked to identify their health status and medication intake, ethnicity or race, age, and sex, and a dental examination was used to determine oral health status either as DMFS, DMF or DFT. Additionally, saliva samples were collected. Information collected from the questionnaire and dental exams was recorded. Saliva samples were analyzed and data obtained from them was correlated with the information recorded from the questionnaire and dental exam. Excess samples obtained from saliva collections was catalogued and frozen at −70° C. indefinitely. Initial correlations were conducted without bias.

### Example 2

### Computing Power and Sample Size

The simple correlation between MUC7 mucin concentration and DFT had a test power of 1.00 with α=0.05 for the group of sixteen subjects minus the outliers. The test power for the remaining four-member outlier group was 0.90. The test power for the total group of twenty subjects was only 0.74, which is below the usually acceptable level of 0.80. However, when age is added to the multiple regression analysis, the power jumps to 1.00. Thus, a larger sample size is not needed for confirming the simple relationship between DFT and MUC7 mucin concentration in whole saliva.

### Example 3

### DMF Correlation With MUC7 Mucin Concentration

Table 3 presents the correlation between MUC7 mucin (MG2) concentration in unstimulated saliva (i.e., the independent variable) and the corresponding DMF (i.e., the dependent variable). The data presented in Table 3 includes all subjects, and gives an adjusted r² value of 0.209, which is quite low, indicating that not a very large amount of the DMF variation can be explained by the mucin variation. The table shows a p value of 0.025, which is below the usual 0.05 needed, and thus establishes that there is a significant relationship between MUC7 mucin concentration and DMF. Mucin concentration used in this regression analysis and saliva flow rates have previously been reported. (Navazesh et al., “Comparison of whole saliva flow rates and mucin concentrations in healthy Caucasian young and aged adults,” J. Dent. Res. 71: 1275-1278 (1992)).

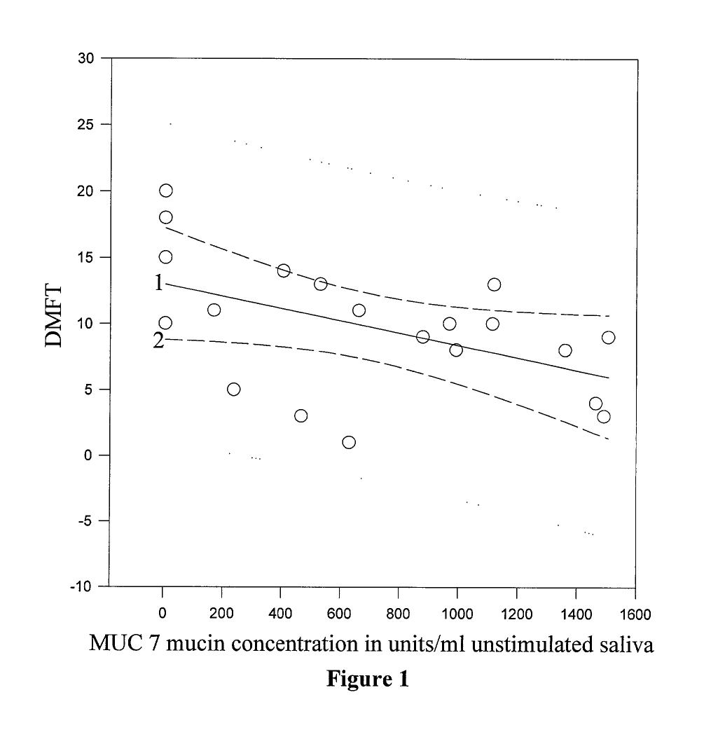

FIG. 1 describes the corresponding graphic plot for the relationship of MUC7 mucin concentration in unstimulated saliva (i.e., the independent variable) with DMF as the dependent variable. As shown in FIG. 1, the slope of the line (1) and the width of the prediction confidence interval (2) provide significant factors for tracking the two iterations of the data. In this figure, the slope is relatively shallow, and the prediction interval too broad to be very useful for using mucin concentration to predict DMF.

TABLE 3
Correlation between MUC7 mucin concentration and DMF

| | r(correlation | r²(coefficient of | |
|---|---|---|---|
| Sample | coefficient) | determination) | p(significance) |
| Group of 20 young | −0.5008 | 0.2092 | 0.02450 |
| adults | | | |

### example 4

### DFT Correlation With MUC7 Mucin Concentration

Table 4 presents the correlation between MUC7 mucin (MG2) concentration in unstimulated saliva (i.e., the independent variable) with the corresponding DFT (i.e., the dependent variable). The missing teeth variable was eliminated because loss of teeth from decay in young adults is rare, whereas loss by trauma is common. Thus, missing teeth are likely to be irrelevant to caries experience in this group of young adults. This regression analysis gave a slight improvement in adjusted r² (0.275), and doubled the significance (0.01033).

TABLE 4
Correlation between MUC7 mucin concentration and DFT

| | r(correlation | r²(coefficient of | |
|---|---|---|---|
| Sample | coefficient) | determination) | p(significance) |
| Group of 20 young | −0.5594 | 0.2748 | 0.01033 |
| adults | | | |

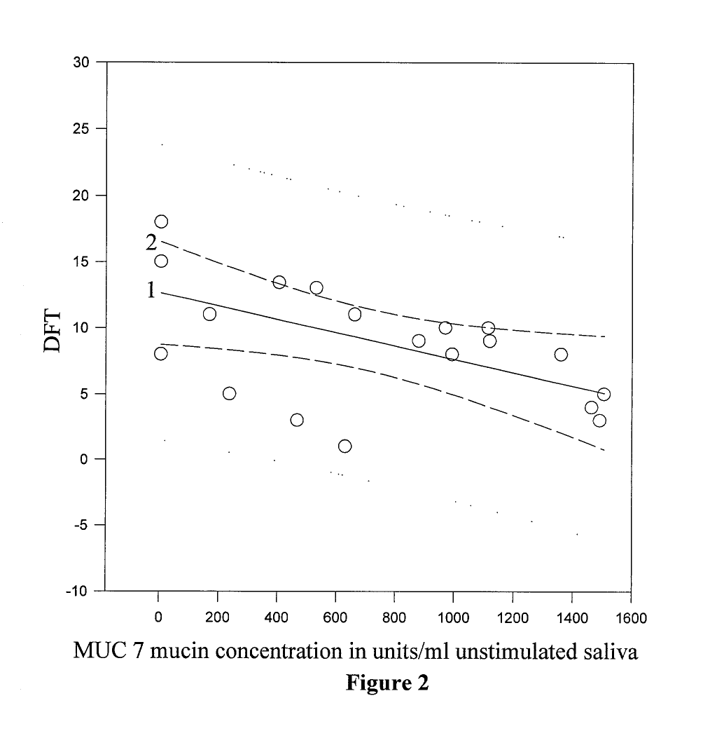

FIG. 2 describes the corresponding graphic plot of the linear regression analysis for the relationship of MUC7 mucin concentration in unstimulated saliva with DFT as the dependent variable. This graph looks very similar to the plot shown in FIG. 1, except that the points are clustering a little tighter around the line. FIG. 3, the corresponding “scatter plot” for the data in Table 4, strongly suggests that there is a subgroup within the total group. This subgroup represents four of the youngest males in the study (referred to as the “outliers”).

Table 5 presents the correlation between MUC7 mucin concentration in unstimulated saliva (i.e., the independent variable) and the corresponding DFT as the dependent variable. This table is similar to Table 4, except the subgroup of four outliers was removed, leaving sixteen subjects. The adjusted r² increased from 0.2571 to 0.8458, and the significance becomes extremely high, with a value <0.0000003. The corresponding graphic plot in FIG. 4 also shows some dramatic changes.

TABLE 5
Correlation between MUC7 mucin concentration and DFT
in young adults without the four member outlier group

| | r(correlation | r²(coefficient of | |
|---|---|---|---|
| Sample | coefficient) | determination) | p(significance) |
| Group of 16 young | −0.9252 | 0.8458 | 0.0000002866 |
| adults | | | |

FIG. 4 describes a linear regression analysis for the relationship of MUC7 mucin concentration in unstimulated saliva (i.e., the independent variable) with DFT as the dependent variable after removing the four outliers shown in FIG. 3. As shown in FIG. 4, the slope has steepened, and the prediction confidence interval narrowed. Together, these changes make it possible to graphically demonstrate that the range of relationships of mucin to DFT can be statistically divided with >95% confidence intervals into at least four significantly different regions of the regression line, 3a, 3b, 3c and 3d. More importantly, this result indicates that every mucin concentration point within the normally distributed continuum predicts a limited range of DFT, approximately three wide (DFT center point ±1) in the mid-section of the regression line and a range of four at either end (DFT center point ±1.5).

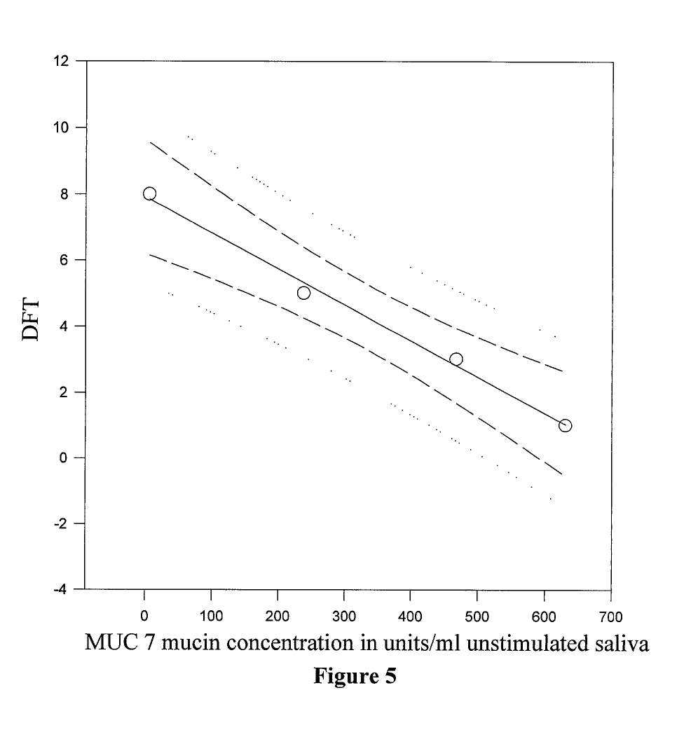

Table 6 presents the correlation for the outliner group, showing the adjusted r² of 0.991 and p value of 0.00302 for just the four samples. FIG. 5 illustrates the corresponding linear regression analysis for the relationship of MUC7 mucin concentration in unstimulated saliva (i.e., the independent variable) with DFT as the dependent variable for the four outliers shown in FIG. 3. As shown in FIG. 5, the slope for the outlier group is fairly similar to that for the larger group of 16 (FIG. 4), whereas the constants at zero concentration of mucin are very different (i.e., 16.1442 vs. 7.915 at zero mucin).

TABLE 6
Correlation between MUC7 mucin concentration
and DFT in the young adult outlier group

| | r(correlation | r²(coefficient of | |
|---|---|---|---|
| Sample | coefficient) | determination) | p(significance) |
| Four member outlier | −0.9970 | 0.9910 | 0.003021 |
| group | | | |

### Example 5

### DFT/DMF Correlation With MUC7 Concentration

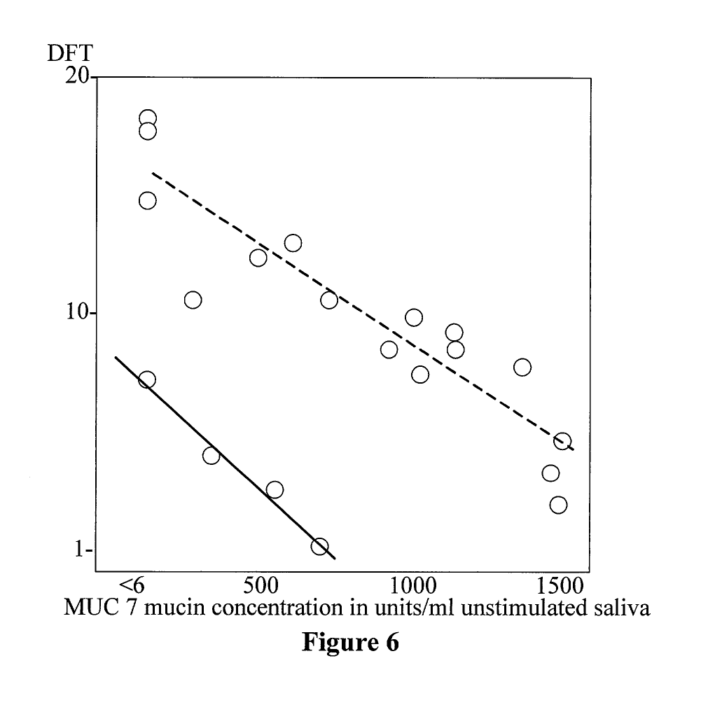

The correlation of DMF to MUC7 mucin concentration in unstimulated saliva within the young adult group, was significant with a p value of 0.025 and an adjusted r² (coefficient of determination) of 0.209. Eliminating missing teeth as a variable and simplifying past caries experience to DFT, resulted in improved statistics (p=0.0103 and r²=0.275). A scatter matrix of this data indicated that there was a group of four young males whose data points were clearly outliers from the other points (FIG. 6). In this figure, the regression lines for the two groups have been superimposed. The line defining the mucin concentration from the outlier group line is solid, while the line defining the mucin concentration from other subjects is broken.

Separate analysis of this small group also showed a significant correlation of DFT with MUC7 mucin concentration in unstimulated saliva, p=0.00302 and r²=0.991. The significance of the correlation in the remaining group of 16 improved to p=0.00000029 and r²=0.846 (Table 1). These correlations were unexpected, in view of recent reviews indicating that cariogenesis is multifactoral, with no single factor that can be viewed as having a broad, unified impact on the initiation of caries in individuals, excluding fluoride exposure (Lenander-Lumikari, M. and Loimaranta, V., Adv. Dent. Res. 14:40-47, 2000). Interestingly, the slopes of the regression lines are similar (−0.0074 [n=16] vs. −0.0109 [n=4]), with the larger difference between the two subgroups residing with the zero mucin intercept constant of 16.2 and 7.9 DFT, respectively.

Thus, a very accurate linear regression line can be obtained using single saliva samples from a relatively small collection of individuals, as long as normal distribution rules are satisfied. However, to set the actual confidence limits for a prediction based on that regression, the extent of normal individual variation becomes important. To explore this aspect of the prognostic capabilities resident in a single saliva sample, the CV's (i.e., coefficients of variation) from a repeated measures study representative of the total 20 member group were used. These CV's were “attached” to mucin concentration values in the correlation study that are similar to the means from the repeated measures study. After calculating the corresponding SDs (standard deviations), the maximum range of the hypothetical mucin mean for individual data points of the correlation study was calculated for 95% confidence limits using the following formula for determining sample sizes: d=Z σ/\sqrt{n}. These ranges were superimposed on the above regression line, which was then used to graphically convert them into ranges of DFTs.

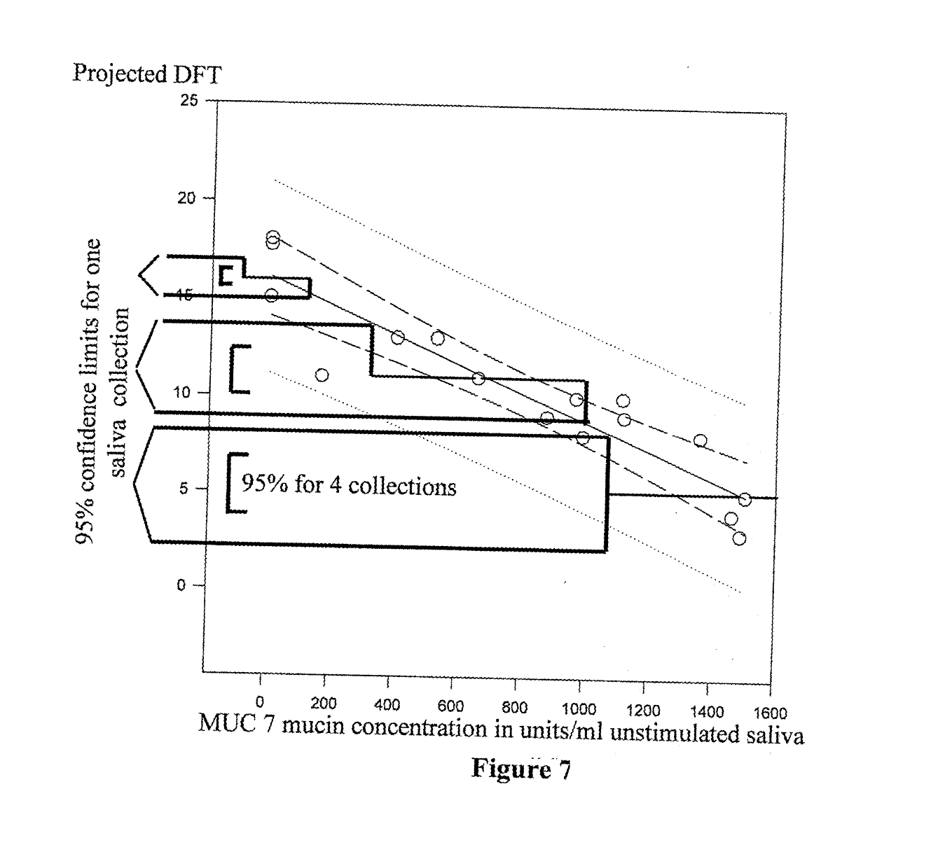

FIG. 7 shows representative 95% confidence intervals for DFT predicted by either one or four saliva collections. In this figure, the regions bracketed by bold solid lines represent the range of DFT, based on CVs from repeated measures, projected by mucin concentration either in one sample or from the mean of four sequential samples of saliva. The mid-range DFT projection uses the greatest CV (i.e., 26.6%), from the repeated measures study. The CV-derived range at low mucin concentrations did not appear to account for the DFT variation at zero mucin concentration. Though the relationship of MUC7 mucin concentration to DFT appears to be a continuum, FIG. 7 graphically shows that there is the possibility of three statistically distinct groups from single saliva samples that can be characterized as high, medium, and low caries experience.

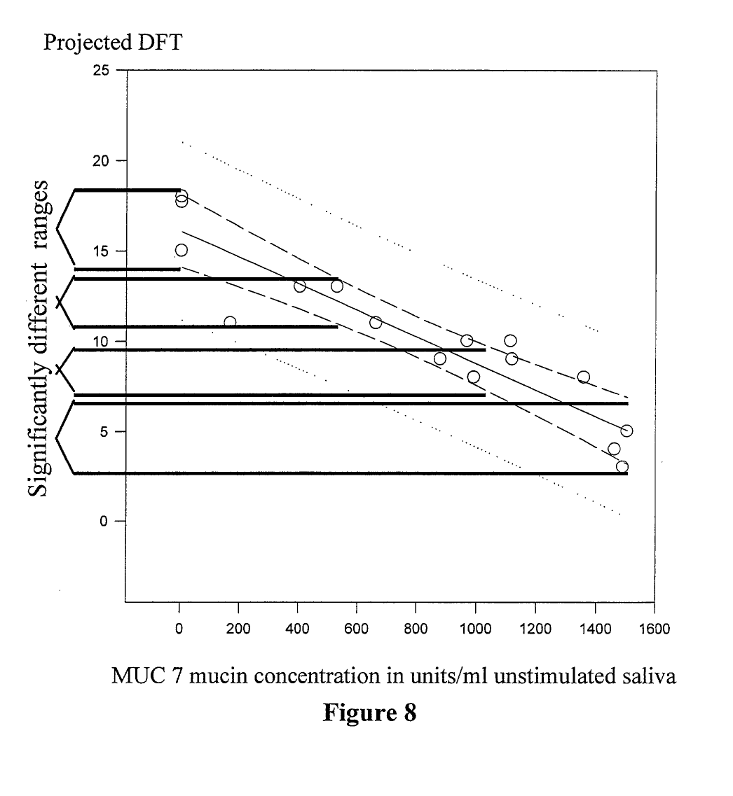

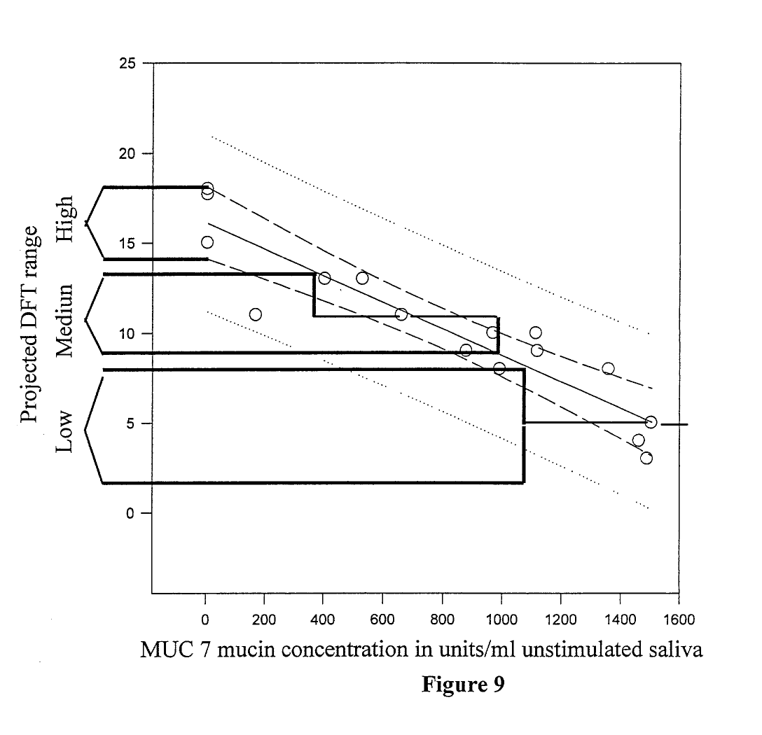

A different prognostic pattern is suggested from the 95% confidence intervals of the regression analysis (FIG. 8), indicating that there is the potential for a total of four significant DFT ranges. FIG. 8 shows significant ranges of DFT projection (A-D) by mucin concentration based solely on 95% confidence intervals of the regression equation. It is noted that individual variation is not fully accounted for in this model, especially at high and medium mucin concentrations. Also, the regression diagram shown in this figure is similar to that shown in FIGS. 7 and 9.

The comparison of FIG. 7 and FIG. 8 suggests that better predictions will be accomplished by a combination of the two approaches, wherein individual variation is the determinate at higher mucin concentrations, and the 95% confidence interval of the regression equation is determinate at low concentrations. FIG. 9 shows that even when the largest CV from the repeated measures study is used at a mid-range mucin concentration, the combination approach still creates a significant three-level test range. The apparent continuum of mucin concentrations indicates that there will be legitimate borderline individuals. This situation will be discussed in a subsequent section along with a strategy for identifying these individuals.

### Example 6

### Multiple Regression Analysis Involving Gender and Age as Additional Variable

Some surveys have reported that in general, females have more DMFS than do males. In comparison, the study using the total group of 20 shows that when the male and female subgroups are age equivalent, males have an average of 9.0 DFT vs. 13.4 DFT for females (p=0.034). The average mucin concentration from this study is higher in males, 977 units per ml vs. 393 units per ml in females (p=0.038). Separate linear regression analyses of the two genders, while showing similar slopes and constants, still show a >10% difference from one another. However, when age is then added as an independent variable to multiple linear regression analyses, the mucin concentration slopes become virtually identical, −0.004974 vs. −0.005021 (<1% difference) for males and females respectively.

Larger sample sizes allow further in-depth examination of gender differences and their potential impact on the single saliva sample test model. Multiple linear regression analyses on the group of sixteen subjects boosted the r² value from 0.846 to 0.902 by including age as one of the independent variables (Table 1). As expected because of identical gender-based regression equations, the further addition of gender to this regression analysis did not change the r² value.

When the concentration of MUC5B mucin (MG1) in unstimulated saliva was also considered as an independent variable, the adjusted r² value increased further to 0.930. This establishes that MUC5B mucin is also a significant predictor of the risk of disease.

The most remarkable improvement occurred when additional independent variables were factored into the regression analysis for the total group of twenty, which includes the outlier group of four. The original adjusted r² of MUC7 mucin concentration in unstimulated saliva vs. DFT was 0.275, but when age is included in the multiple linear regression analysis, the r² value increases to 0.709 (Table 1). Inclusion of gender at this point of the analysis raised the r² value to 0.773, while including unstimulated MUC5B further increases r² to 0.789.

### Example 7

### Tests for Total Apoprotein, Carbohydrate, and Sialic Acid Content in Salivary Mucins

The SDS-PAGE method described above, is adapatable for quantitating total apomucin and total carbohydrate content of salivary mucins.

SDS-PAGE quantitation uses appropriate amounts of a saliva sample, which are fractionated by SDS-PAGE on three separate lanes of a slab gel. For apoprotein quantitation, one lane is treated with a combination of Coomassie Blue and silver stain (Culp, D. J., Latchney, L. R., Frampton, M. W., Jahnke, M. R., Morrow, P. E., and Utell, M. J. Lung Cell. Mol. Physiol. 269:L358-L370, 1995). Total carbohydrate is quantitated from the second lane by ALCIAN BLUE® staining following conversion of all monosaccharides, neutral and acidic, to sulfonates (Culp et al., supra). The third lane containing the fractionated mucins, could be used to quantitate total sialic acid by the STAINS-ALL® method (Denny et al., 1991, Baughan et al., 2000). Previously used models for establishing test linearity, and within- and between-test variation (Denny et al, supra) are followed.

One object of these tests is to accurately assess relative differences between subjects, so that absolute quantitation may not always be necessary. Thus, the most practical standard for comparison may be a pooled sample from the salivas of several individuals, with which all other individual saliva samples will be compared. The pooled sample may routinely be monitored for stability with a heterologous pure acidic glycoprotein standard, such as fetuin. In one embodiment, the quantitation of the three tests (i.e., total apomucin, total carbohydrate, and total sialic acid) will be performed using video images or direct scanning, which has been color-adjusted to maximize the linearity of each stain, and then imported into a densiometric program, such as SIGMASCAN® so that the band intensities can be quantitated (Denny et al., 1991). In addition, it is contemplated that the SDS-PAGE tests for the three components of MUC7 (i.e., total apoprotein, total carbohydrate, and total sialic acid) can be used to provide similar data for other salivary mucins, such as MUC5B mucin, in the same lanes of the SDS-PAGE gel.

### Example 8

### High Throughput Tests Employing MUC7 Antibodies

In one embodiment, a high throughput test uses a mucin capture mechanism that employs the direct surface of a multiwell plate (Bolscher, J. G. M., Grownink, J., van der Kwaak, J. S., van den Keijbus, P. A. M., van 't Hof, W., Veerman, E. C. I., and Nieuw Amerongen, A. V. J. Dent. Res. 78:1362-1369, 1999). Alternatively, a high throughput test uses a mucin capture mechanism that employs an antibody to a mucin, such as MUC7 mucin, that has first interacted to the surface of a multiwell plate (Rayment, S. A., Liu, B., Offner, G. D., Oppenheim, F. G., and Troxler, R. F. J. Dent. Res. 79:1765-1772, 2000). Furthermore, fluorescent or chemiluminescent reporters are used for assaying each fraction of the mucin is contemplated. For the apomucin, an antibody directed to a non-glycosylated C-terminal domain of a mucin, such as MUC7 mucin, will be used for the initial capture (Bolscher et al., supra). An antibody derivitized to trigger fluorescent or chemiluminescent reporters and directed to a non-glycosylated N-terminal mucin domain (Rayment et al., 2000) will be used to quantitate the captured mucin. Such antibodies can be produced commercially from synthetic peptides (Bolscher et al, 1999; Rayment et al, 2000).

### Example 9

### High Throughput Tests for Quantitation of Mucin Carbohydrates

Several approaches are available for measuring the amount of total carbohydrates in salivary mucins. In one embodiment, different biotinylated lectins are used with an isolated mucin. Although WGA has been used in an ELISA to quantitate MUC7 mucin (Rayment et al., 2000), its primary affinity for glucosamine with only secondary affinity for sialic acid does not appear to be a property that represents the core carbohydrates of MUC7 mucin (Prakobphol, A., Tangemann, K., Rosen, S. D., Hoover, C. I., Leffler, H., and Fisher, S. J., Biochemistry 38:6817-6825, 1999). The lectins, BPL, PNA, or RCA I after desialylation are more appropriate for core carbohydrates. These detect the “asialo-T-antigen” that is involved along with its sialylated version in a mucin, such as the MUC7 mucin, bound to oral microbes (Prakobphol et al., 1999). The lectins, Jacalin and ACL also recognize the sialylated T-antigen. Lectins can also be used in tests that rely on recognition of the Lewis antigens of salivary mucins. For example, the Lewis antigen of MUC7 mucin, which binds neutrophils (Prakobphol et al., 1999), is recognized by AAL, LTL, and UEA I. Thus, the use of lectins provides an analytical tool for precisely identifying a mucin carbohydrate that gives the best predictive power with the lowest individual CVs. The ELISA-light™ system can be used for quantitation. Beacause the antibodies used to capture mucins also contain carbohydrate, steps are taken to control their effect.

### Example 10

### High Throughput Tests for Quantitation of Total Sialic Acid

Several tests are available for quantifying the total amount of sialic acid in a salivary mucin. For example, a biotinylated lectin that is directed at the terminal sialic acids of the captured mucin in a multiwell plate can be used. A lectin commonly used for this purpose is SNA, which preferentially binds α-2,6 linked sialic acids. Other lectins are used depending on the particular salivary mucin being analyzed. For example, because the two most prevalent oligosaccharides of MUC7 mucin have sialic acid in the α-2,3 position (Prakobphol et al. supra), biotinylated MAL II lectin, which favors this linkage, is preferred for MUC7 mucin. The ELISA-light system is again be used for quantitation. All these tests are calibrated against a battery of salivas whose range of caries prediction has been verified by the SDS-PAGE method and includes the full range of DFT in the study group. The emphasis on the use of lectins for the carbohydrate assays is predicated on the opportunity for “fine-tuning” the moiety of a mucin that most accurately forecasts a disease. In another embodiment, total carbohydrates and sialic acid are measured using the PAS and thiobarbituric acid colorimetric assays, respectively.

### Example 11

### Predicting Caries Risk of Individuals Within a Particular Age Group

A statistically significant contribution to the predictive power of the present invention was achieved when using age as one of the independent variables in multiple linear regression analysis (Table 1). The age group ranging from 18 to 35 years of age was studied. Within this group, individuals were measured for the amount of variation of the MUC7 mucin sialic acid concentration in saliva at weekly intervals. Coefficients of variation (“CV”) for these individuals ranged between 13% and 26%, with a mean of 20%. The average CV is useful for establishing the confidence intervals of the risk prediction, and must also be age-appropriate to provide an accurate prediction. Furthermore, age alone (Table 2), as well as in combination with other factors (Table 1), has been a significant contributor to caries forcasting. Thus, this invention recognizes the importance of age as a factor and acknowledges a consequence that different regression equations may be needed for every age group.

In one embodiment, the same tests as described for MUC7 mucin will be performed in unstimulated saliva collected from children 7-8 years old. This narrow age range is envisioned as one means for diminishing the impact of age on the caries prediction process. The children will receive a simple oral examination to score their DMFS, focusing on their remaining eight primary molars. The parents will be asked to complete a simple questionnaire. These tests will provide the evidence for the expected correlation of MUC7 mucin data from saliva and individual caries experience in children. Significant correlation in these tests also raises the expectation that the test has value as a predictor of future caries risk in children as well as a non-invasive forecaster of accumulated caries experience. These tests will also identify the conditions and variables that support the use of a practical test to provide at least three significantly different ranges of prediction. Variables specific to children may be important, such as the ratio of deciduous to permanent teeth, number of developmentally appropriate missing teeth, and the proportion of healthy teeth as a function of age. In the course of these tests, a custom regression equation will be derived that specific for this age group.

### Example 12

### Development of a Prototype Test for Predicting Caries

It is an object of the present invention to develop a practical test for predicting caries experience from saliva that is easily and accurately interpreted. In one embodiment, the present invention provides a practical test for predicting caries from a single saliva sample of young adults. In a particular embodiment, it is contemplated that a practical test for predicting caries from saliva includes a strip test, analogous to a dot blot test, that can distinguish five mucin concentrations (e.g., equivalent to 100, 450, 800, 1150, and 1500 units per ml).

A strip test provides various advantages over other possible designs in its ease of distribution, use, and interpretation. To explain the rationale for a design of five incremental concentrations, the paradigm is changed to view the mucin concentration continuum range of 0 to 1500 units as having three zones of significance when assayed in a single saliva sample (FIG. 9). However when applied to interpretation of an amount present in the saliva of an individual, the zone boundaries float such that each individual's significant personal zone, which is equivalent to five or six DFT, has two additional significant zones above or below it or is positioned between significant other zones. The potential for a continuum of personal zones clashes with the three-level test featured above because of the reality of personal zones that traverse the hypothetical boundaries between high, medium, and low ranges of caries assessment. The potential for assaying five mucin concentrations on the strip test suggests a solution to the problem in the following manner.

In the mucin concentration continuum, the 450 and 1150 points represent the theoretical boundaries of the three-level test. However, any individual that assays at or near a mucin concentration of 450 units per ml and no higher, will be ranked as high caries potential. Based on the statistics of individual variation, this individual's saliva spends at least 50% of its time within the lowest range of mucin concentrations. The same reasoning would apply to the samples exhibiting near 1150 (i.e., they would be assigned to the middle range of cumulative caries experience/risk). From the 16-subject group minus the outliers of the above study, the 450 mark would place 31% in the high caries potential range. This increases to 35% in the total group, which as noted earlier errors by bumping the four members of the outlier group into the next higher caries group. Other studies based on clinical examination generally regard the caries-prone fraction of the population to be approximately 25% (U.S. Dept. of Health & Human Services. National Institutes of Health Consensus Development Conference Statement. Diagnosis and Management of Dental Caries Throughout Life, (2001). Thus, if a way is found to pre-identify members of the outlier group and subsequently assign them to their appropriate groups, then the proposed mucin concentration cut-offs will have identified 25% of the preliminary study subjects as caries-prone.

The test strip design is also simple. The fundamental design of a five concentration test strip is envisioned to be based on either spotting multiple concentrations of antibody and a single intensity of color to be matched against a standard, or a single spot of antibody and multiple intensities of colors to be matched with a range of standard color intensities. Which approach to be used depends on the kinetics and affinities of various antibody and dye/stain combinations. It is contemplated that the capture antibody will be permanently attached to the strip support either by derivatization or by direct fixation. Various ways of visualizing the amount of mucin captured are contemplated, such as direct binding of specific stains (e.g., alcian blue, silver-enhanced alcian blue, or Stains-All); chromophore-labelled lectins; and various indirect methods, such as enzyme catalyzed amplification. The vast majority of the reagents to be used during development of the test are commercially available. Once the prototype test has been developed, it will be validated with a panel of the existing archived saliva samples from young adults and children.

An alternative to the significant three-level strip test is a two-level strip test predicting high and low caries experience, whose significance could be achieved with less discriminating data. This also introduces the possibility of using multiple logistic regression analysis, which easily handles the caries/caries-free situation and the use of categorical and text-based independent variable information, such as gender and yes/no answers from the questionnaires.

It is understood that the examples and embodiments described herein are for illustrative purposes only and that various modifications or changes in light thereof will be suggested to persons skilled in the art, and are to be included within the spirit and purview of the invention as set forth in the appended claims. All publications and patents cited herein are incorporated herein by reference in their entirety for all purposes.

We claim:

1. A method for predicting the risk of a disease, comprising the steps of:
a) providing a saliva sample from a subject;
b) isolating a mucin in said saliva sample to produce an isolated mucin; and
c) quantitating the content of a component of said isolated mucin to predict the risk of a disease in said subject.

2. The method of claim 1, wherein said saliva sample is a stimulated saliva sample.

3. The method of claim 1, wherein said saliva sample is an unstimulated saliva sample.

4. The method of claim 1, wherein said component is total apomucin.

5. The method of claim 1, wherein said component is total carbohydrate.

6. The method of claim 1, wherein said component is sialic acid.

7. The method of claim 1, wherein said mucin is MUC7 mucin.

8. The method of claim 1, wherein said mucin is MUC5AC mucin.

9. The method of claim 1, wherein said mucin is MUC5B mucin.

10. The method of claim 1, further comprising the step of reporting the content of said component in said isolated mucin as units per milliliter of said saliva sample.

11. The method of claim 1, further comprising the step of assessesing the risk of said disease as high, medium or low.

12. The method of claim 1, further comprising the step of assessing the risk of future development of said disease in said subject.

13. The method of claim 12, further comprising the step of assessing the risk of future development of said disease in said subject at subsequent ages.

14. The method of claim 1, wherein said isolating step comprises isolating said mucin using SDS-PAGE.

15. The method of claim 1, wherein said quantitating step comprises specifically binding said component in said isolated mucin.

16. The method of claim 15, wherein said binding comprises direct binding or facilitated binding.

17. The method of claim 16, wherein said direct binding comprises dye-binding.

18. The method of claim 16, wherein said facilitated binding comprises linking a specifically binding member pair to a surface that specifically binds to said component of said isolated mucin.

19. The method of claim 18, wherein said specifically binding member pair is selected from the group consisting of antibodies and lectins.

20. The method of claim 1, wherein said subject is a human.

21. The method of claim 20, wherein said human is selected from the group consisting of males and females.

22. The method of claim 20, wherein said subject is aged between 18 and 35 years old.

23. The method of claim 1, wherein said disease is selected from the group consisting of dental caries, periodontal diseases, pulmonary diseases, respiratory diseases, cardiovascular diseases, diabetes, perinatal disorders, mucosal infections, oral cancers, pharyngeal cancers, precancerous lesions, associated autoimmune disorders, HIV, osteoporosis, and a combination thereof.

24. The method of claim 23, wherein said periodontal diseases are selected from the group consisting of gingivitis, adult periodontitis, early-onset periodontitis, and a combination thereof.

25. The method of claim 23, wherein said cardiovascular diseases are selected from the group consisting of heart attack, stroke and atherosclerosis.

26. The method of claim 23, wherein said perinatal disorders are selected from the group consisting of low birth weight and preterm births.

27. The method of claim 23, wherein said diabetes is Type 1 diabetes or Type 2 diabetes.

28. The method of claim 23, wherein said mucosal infections are selected from the group consisting of oral candidiasis, herpes simplex virus infections, herpes zoster virus infections, varicella zoster virus infections, human papillomavirus infections, oral human papillomavirus infections, recurrent aphtous ulcers, and combinations thereof.

29. The method of claim 28, wherein said herpes simplex virus is Type 1 or Type 2.

30. The method of claim 23, wherein said disease is dental caries.

31. The method of claim 30, wherein said dental caries is selected from the group consisting of early-onset dental caries, adult dental caries, root caries, DFT, DMF, and DMFS.

32. A method for reducing the risk of a disease, comprising the steps of:
a) providing a saliva sample from a subject;
b) isolating a mucin in said saliva sample to produce an isolated mucin;
c) quantitating the content of a component in said isolated mucin; and
d) administering a therapeutic reagent to said subject when the content of said component in said isolated mucin significantly falls below the level expressed in an oral fluid standard.

33. The method of claim 32, wherein said saliva sample is a stimulated saliva sample.

34. The method of claim 32, wherein said saliva sample is an unstimulated saliva sample.

35. The method of claim 32, wherein said component is total apomucin.

36. The method of claim 32, wherein said component is total carbohydrate.

37. The method of claim 32, wherein said component is sialic acid.

38. The method of claim 32, wherein said mucin is MUC7 mucin.

39. The method of claim 32, wherein said mucin is MUC5AC mucin.

40. The method of claim 32, wherein said mucin is MUC5B mucin.

41. The method of claim 32, wherein said isolating step comprises isolating said mucin using SDS-PAGE.

42. The method of claim 32, wherein said quantitating step comprises specifically binding said component of said isolated mucin.

43. The method of claim 42, wherein said binding comprises direct binding or facilitated binding.

44. The method of claim 43, wherein said direct binding comprises dye-binding.

45. The method of claim 43, wherein said facilitated binding comprises linking a specifically binding member pair to a surface that specifically binds to said component of said isolated mucin.

46. The method of claim 45, wherein said specifically binding member pair is selected from the group consisting of antibodies and lectins.

47. The method of claim 32, wherein said subject is a human.

48. The method of claim 47, wherein said human is selected from the group consisting of males and females.

49. The method of claim 32, wherein said subject is aged from between 18 to 35 years of age.

50. The method of claim 32, wherein said disease is selected from the group consisting of periodontal diseases, pulmonary diseases, respiratory diseases, cardiovascular diseases, diabetes, perinatal disorders, mucosal infections, oral cancers, pharyngeal cancers, precancerous lesions, associated autoimmune disorders, HIV, osteoporosis, and a combination thereof.

51. The method of claim 50, wherein said periodontal diseases are selected from the group consisting of gingivitis, adult periodontitis, early-onset periodontitis, and a combination thereof.

52. The method of claim 50, wherein said cardiovascular diseases are selected from the group consisting of heart attack, stroke and atherosclerosis.

53. The method of claim 50, wherein said perinatal disorders are selected from the group consisting of low birth weight and preterm births.

54. The method of claim 50, wherein said diabetes is Type 1 diabetes or Type 2 diabetes.

55. The method of claim 50, wherein said mucosal infections are selected from the group consisting of oral candidiasis, herpes simplex virus infections, herpes zoster virus infections, varicella zoster virus infections, human papillomavirus infections, oral human papillomavirus infections, recurrent aphtous ulcers, and combinations thereof.

56. The method of claim 55, wherein said herpes simplex virus is Type 1 or Type 2.

57. The method of claim 32, wherein said disease is dental caries.

58. The method of claim 57, wherein said dental caries is selected from the group consisting of early-onset dental caries, adult dental caries, root caries, DFT, DMF, and DMFS.

59. The method of claim 57, wherein said therapeutic reagent is an anti-caries reagent.

60. The method of claim 32, wherein said oral fluid standard comprises a sample from a normal control.

61. A diagnostic kit for detecting a disease comprising:
a) a means for collecting a saliva sample;
b) a means for isolating a mucin in said saliva sample, to produce an isolated mucin;
c) a means for measuring the amount of a component in said isolated mucin; and
d) an oral fluid standard for comparing the amount of said component in said isolated mucin.

62. The method of claim 61, wherein said oral fluid standard comprises the content of said component in said mucin of a normal subject.

63. The diagnostic kit of claim 61, wherein said disease is selected from the group consisting of dental caries, periodontal diseases, pulmonary diseases, respiratory diseases, cardiovascular diseases, diabetes, perinatal disorders, mucosal infections, oral cancers, pharyngeal cancers, precancerous lesions, associated autoimmune disorders, HIV, osteoporosis, and a combination thereof.

64. The diagnostic kit of claim 63, wherein said periodontal diseases are selected from the group consisting of gingivitis, adult periodontitis, early-onset periodontitis, and a combination thereof.

65. The diagnostic kit of claim 63, wherein said cardiovascular diseases are selected from the group consisting of heart attack, stroke and atherosclerosis.

66. The diagnostic kit of claim 63, wherein said perinatal disorders are selected from the group consisting of low birth weight and preterm births.

67. The diagnostic kit of claim 63, wherein said diabetes is Type 1 diabetes or Type 2 diabetes.

68. The diagnostic kit of claim 63, wherein said mucosal infections are selected from the group consisting of oral candidiasis, herpes simplex virus infections, herpes zoster virus infections, varicella zoster virus infections, human papillomavirus infections, oral human papillomavirus infections, recurrent aphtous ulcers, and combinations thereof.

69. The diagnostic kit of claim 68, wherein said herpes simplex virus is Type 1 or Type 2.

70. The diagnostic kit of claim 63, wherein said disease is dental caries.

71. The diagnostic kit of claim 69, wherein said dental caries is selected from the group consisting of early-onset dental caries, adult dental caries, root caries, DFT, DMF, and DMFS.
